SEQUENCE LISTING (48 sequences)

SEQ ID NO: 1 - DNA, 2376 bp, Homo sapiens
gaggaggagtggggaccgggcggggggtggaggaagaggcctcgcgcagaggagggagcaattgaatttcaaacacaaacaactcgacgagcgcgcacccaccgcgccggagccttgccccgatccgcgcccgccccgtccgtgcggcgcgcgggcggagacgccgtggccgcgccggagctcgggccgggggccaccatcgaggcgggggccgcgcgagggccggagcggagcggcgccgccaccgccgcacgcgcaaacttgggctcgcgcttcccggcccggcgcggagcccggggcgcccggagccccgccatgtcgcgatccaaccggcagaaggagtacaaatgcggggacctggtgttcgccaagatgaagggctacccacactggccggcccggattgacgagatgcctgaggctgccgtgaaatcaacagccaacaaataccaagtcttttttttcgggacccacgagacggcattcctgggccccaaagacctcttcccttacgaggaatccaaggagaagtttggcaagcccaacaagaggaaagggttcagcgaggggctgtgggagatcgagaacaaccctactgtcaaggcttccggctatcagtcctcccagaaaaagagctgtgtggaagagcctgaaccagagcccgaagctgcagagggtgacggtgataagaaggggaatgcagagggcagcagcgacgaggaagggaagctggtcattgatgagccagccaaggagaagaacgagaaaggagcgttgaagaggagagcaggggacttgctggaggactctcctaaacgtcccaaggaggcagaaaaccctgaaggagaggagaaggaggcagccaccttggaggttgagaggccccttcctatggaggtggaaaagaatagcaccccctctgagcccggctctggccgggggcctccccaagaggaagaagaagaggaggatgaagaggaagaggctaccaaggaagatgctgaggccccaggcatcagagatcatgagagcctgtagccaccaatgtttcaagaggagcccccaccctgttcctgctgctgtctgggtgctactggggaaactggccatggcctgcaaactgggaacccctttcccaccccaacctgctctcctcttctactcacttttcccactccaagcccagcccatggagattgacctggatggggcaggccacctggctctcacctctaggtccccatactcctatgatctgagtcagagccatgtcttctccctggaatgagttgaggccactgtgttccttccgcttggagctattttccaggcttctgctggggcctgggacaactgctcccacctcctgacacccttctcccactctcctaggcattctggacctctgggttgggatcaggggtaggaatggaaggatggagcatcaacagcagggtgggcttgtggggcctgggaggggcaatcctcaaatgcggggtgggggcagcacaggagggcggcctccttctgagctcctgtcccctgctacacctattatcccagctgcctagattcagggaaagtgggacagcttgtaggggaggggctcctttccataaatccttgatgattgacaacacccatttttccttttgccgaccccaagagttttgggagttgtagttaatcatcaagagaatttggggcttccaagttgttcgggccaaggacctgagacctgaagggttgactttacccatttgggtgggagtgttgagcatctgtccccctttagatctctgaagccacaaataggatgcttgggaagactcctagctgtcctttttcctctccacacagtgctcaaggccagcttatagtcatatatatcacccagacataaaggaaaagacacattttttaggaaatgtttttaataaaagaaaattacaaaaaaaaattttaaagacccctaaccctttgtgtgctctccattctgctccttccccatcgttgcccccatttctgaggtgcactgggaggctccccttctatttggggcttgatgactttctttttgtagctggggctttgatgttccttccagtgtcatttctcatccacataccctgacctggccccctcagtgttgtcaccagatctgatttgtaacccactgagaggacagagagaaataagtgccctctcccaccctcttcctactggtctctctatgcctctctacagtctcgtctcttttaccctggcccctctcccttgggctctgatgaaaaattgctgactgtagctttggaagtttagctctgagaaccgtagatgatttcagttctaggaaaataaaacccgttgattact

SEQ ID NO: 2 - protein, 240 aa, Homo sapiens
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCVEEPEPEPEAAEGDGDKKGNAEGSSDEEGKLVIDEPAKEKNEKGALKRRAGDLLEDSPKRPKEAENPEGEEKEAATLEVERPLPMEVEKNSTPSEPGSGRGPPQEEEEEEDEEEEATKEDAEAPGIRDHESL

SEQ ID NO: 3 - protein, 304 aa, Pan troglodytes
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCVEEPEPEPEAAEGDGDKKGNAEGSSDEEGKLVIDEPAKEKNEKGALKRRAGDLLEDSPKRPKEAENPEGEEKEAAQEGSLLLSSCPLLHLLSQLPRFRESGTACRGGAPFHKSLMIDNTHFSFCRPQEKSTGVAGEGTLRAHVLIIRSAAESGEHAGKTPLRDAHQQHLQIVEGKKGRISGACKLLWAGVVQTWID

SEQ ID NO: 4 - protein, 230 aa, Canis familiaris
MGRAQLVLGLPGGRGRTSIDEMPEAAVKSTANKYQVFFFGTHETAFLGPKDLFPYEESKEKFGKPNKRKGFSEGLWEIENNPTVKASGYQSTPKKSSGDEPEPEPEAAEGDGDKKGNAEGSSDEEGKLVIDEPAKEKNEKGALKRRAGELLEDSPKRPKEAEDPEEEEKEVASLEGERPLPVEVEKNSTPSEPSSGRGPPQEEEEEEEEEEEEAAKEDAEAPGIRDHESL

SEQ ID NO: 5 - protein, 237 aa, Mus musculus
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCAAEPEVEPEDHEGDGDKKGSAEGSSDEEGKLVIDEPAKEKNEKGTLKRRAGDVLEDSPKRPKESGDHEEEDKEIAALEGERPLPVEVEKNSTPSEPDSGQGPPAEEEEGEEEAAKEEAEAQGVRDHESL

SEQ ID NO: 6 - protein, 237 aa, Rattus norvegicus
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCAEEPEVEPEAHEGDGDKKGNAEGSSDEEGKLVIDEPAKEKNEKGMLKRRAGDMLEDSPKRPKESGDHEEEEKEIAALEGERPLPVEMEKNSTPSEPDSGQGPPPEEEEGEEEAAKEEAEAQGVRDHESL

SEQ ID NO: 7 - protein, 239 aa, Bos taurus
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCVEEPEPEPEATEGDGDKKGNAEGSSDEEGKLVIDEPTKEKNEKGALKRRAGDLLEDSPKRPKEAEDLEGEEKEGATLEGERPLPVEAEKNSTPSEPGSGRGPPQEEEEEEEEEEAAKEDAEAPGLRDHESL

SEQ ID NO: 8 - DNA, 1333 bp, Mus musculus
ggccaaggagggggcgcccagagcacacgctatgtcttgcttcagccgctctaagtacaagaccggggacctggtgtttgccaagttaaagggctatgcccactggccagcgaggatcgaacatgtcgctgaagccaaccgctaccaggtgttcttcttcgggacccatgagaccgccctgctgggtcccaggcacctgttcccttatgaggagtccaaagagaagttcggcaagcccaacaagaggcgcggcttcagcgaggggctgtgggagatcgagcacgaccctatggttgaggcctcctctagcctgtgctcagaagaggatcagagctacacagaggatcctgggctagcagaggagccggaactaggacaggagctggtgcaggagctggagcccgaattcatgcctgagctggaggcagaacctgagatgcctgagaccgagtgtgaacaggagcccgagccccagcctgcctatgacctgctggacgccgtggaggagccgggcctcaccaaggccgagccaggagatcagcaagccgagcatgtgcaagagaagcaccctgaggtggaggcagaggctgaggctgaggctgaggccgaggcagaggcggaggcggaggccaaggctgaggtggaggagccggggagtctgaagaggagcgcggaggatgaagagcctcattgtcctctcaaacggcccagggaggcggctcctggtgcgttggagatggagcctgctgaagagcgcgaggctgaggcctcccccttcgtggaggagcctgaccaagcccaggagcagctgcctccgttggaggaagaggccacagaaaaggcggtccagggcctgattgttggagaaatagaaggcctgtagtcacggtgtttgtagaagagccccttttacccgttcctgctgccacctggctgtggcttgggaaacccactagggcccagtcttcaacccagtccacctctccttcattcattcgccttctacaagcctaaattcctggcatgaggggacaggccagttgacatcaactttgtggcttaagtagtgaccacccttcccaccctaaactcttaggggcattttggacctctgcctaggaaccaggagtgaagagggatggaagatgaagagcagggacaacattcaaaggtctggaaggaacagtctgtaaactatggcgatgctagtctactccacacccaccctgagtgccgcccataattcctttcagagtactgtaggttcggggaaagtgagacagcagtgtccagtaaagatttttgttgtcccagtcactttctttctttcccg

SEQ ID NO: 9 - protein, 283 aa, Mus musculus
MSCFSRSKYKTGDLVFAKLKGYAHWPARIEHVAEANRYQVFFFGTHETALLGPRHLFPYEESKEKFGKPNKRRGFSEGLWEIEHDPMVEASSSLCSEEDQSYTEDPGLAEEPELGQELVQELEPEFMPELEAEPEMPETECEQEPEPQPAYDLLDAVEEPGLTKAEPGDQQAEHVQEKHPEVEAEAEAEAEAEAEAEAEAKAEVEEPGSLKRSAEDEEPHCPLKRPREAAPGALEMEPAEEREAEASPFVEEPDQAQEQLPPLEEEATEKAVQGLIVGEIEGL

SEQ ID NO: 10 - protein, 272 aa, Canis familiaris
MSRCCRRKYESGDLHLVEPNRYQVFFFGTHETAFLGPKHLFPYEESKEKFGKPSKRRSFSEGLWEIENNPSVRAAADPPAPQEQRGAASAGAGAGEEQLQREEELQREEQQREEQLQREEQLQREEELQREEQLQREEQLQREEEQLHRAEQARAKRSAGELPEGAPKRPREAEPERDIWATQQPYDLDAAIIHIVLARKLRCGDVRGLTKLVTGVTRAPVHCWSLPRIADRLCFLVADGSALARNGEPWSLGELSIESQFISVASLATWRL

SEQ ID NO: 11 - protein, 304 aa, Rattus norvegicus
MSCFSRPKYKTGDLVFAKLKGYAHWPARIEHVTEPNRYQVFFFGTHETALLGPKHLFPYEESKERFGKPNKRRGFSEGLWEIEHDPMVEASPCLCPDEEQLCAEEPGPGEEPEPGQELEPESRPELESMPELEAEPRPEKECEQEPEQEPEQEPEQELEQEPELEPEPEPEPEPEPEPEPEPEPQPAYDLLDAKEEPGLIEAEPGDQQAEQVREQHAEAEVMAVVEEPESLKRSAEDEQPHSPPKRPREAAPGALEMEPAGEREAEACPFVEEPDQAQEQQTPLEEEATEEAVQGLMVGEIEGL

SEQ ID NO: 12 - DNA, 2344 bp, Homo sapiens
cccggcttcgcctcccgctgccgccgccgccgccgccgcagccgctaccgccgctgcagccgctttccgcggcctgggcctctcgccgtcagcatgccacacgccttcaagcccggggacttggtgttcgctaagatgaagggctaccctcactggcctgccaggatcgacgacatcgcggatggcgccgtgaagcccccacccaacaagtaccccatctttttctttggcacacacgaaacagccttcctgggacccaaggacctgttcccctacgacaaatgtaaagacaagtacgggaagcccaacaagaggaaaggcttcaatgaagggctgtgggagatccagaacaacccccacgccagctacagcgcccctccgccagtgagctcctccgacagcgaggcccccgaggccaaccccgccgacggcagtgacgctgacgaggacgatgaggaccggggggtcatggccgtcacagcggtaaccgccacagctgccagcgacaggatggagagcgactcagactcagacaagagtagcgacaacagtggcctgaagaggaagacgcctgcgctaaagatgtcggtctcgaaacgagcccgaaaggcctccagcgacctggatcaggccagcgtgtccccatccgaagaggagaactcggaaagctcatctgagtcggagaagaccagcgaccaggacttcacacctgagaagaaagcagcggtccgggcgccacggaggggccctctggggggacggaaaaaaaagaaggcgccatcagcctccgactccgactccaaggccgattcggacggggccaagcctgagccggtggccatggcgcggtcggcgtcctcctcctcctcttcctcctcctcctccgactccgatgtgtctgtgaagaagcctccgaggggcaggaagccagcggagaagcctctcccgaagccgcgagggcggaaaccgaagcctgaacggcctccgtccagctccagcagtgacagtgacagcgacgaggtggaccgcatcagtgagtggaagcggcgggacgaggcgcggaggcgcgagctggaggcccggcggcggcgagagcaggaggaggagctgcggcgcctgcgggagcaggagaaggaggagaaggagcggaggcgcgagcgggccgaccgcggggaggctgagcggggcagcggcggcagcagcggggacgagctcagggaggacgatgagcccgtcaagaagcggggacgcaagggccggggccggggtcccccgtcctcctctgactccgagcccgaggccgagctggagagagaggccaagaaatcagcgaagaagccgcagtcctcaagcacagagcccgccaggaaacctggccagaaggagaagagagtgcggcccgaggagaagcaacaagccaagcccgtgaaggtggagcggacccggaagcggtccgagggcttctcgatggacaggaaggtagagaagaagaaagagccctccgtggaggagaagctgcagaagctgcacagtgagatcaagtttgccctaaaggtcgacagcccggacgtgaagaggtgcctgaatgccctagaggagctgggaaccctgcaggtgacctctcagatcctccagaagaacacagacgtggtggccaccttgaagaagattcgccgttacaaagcgaacaaggacgtaatggagaaggcagcagaagtctatacccggctcaagtcgcgggtcctcggcccaaagatcgaggcggtgcagaaagtgaacaaggctgggatggagaaggagaaggccgaggagaagctggccggggaggagctggccggggaggaggccccccaggagaaggcggaggacaagcccagcaccgatctctcagccccagtgaatggcgaggccacatcacagaagggggagagcgcagaggacaaggagcacgaggagggtcgggactcggaggaggggccaaggtgtggctcctctgaagacctgcacgacagcgtacgggagggtcccgacctggacaggcctgggagcgaccggcaggagcgcgagagggcacggggggactcggaggccctggacgaggagagctgagccgcgggcagccaggcccagcccccgcccgagctcaggctgcccctctccttccccggctcgcaggagagcagagcagagaactgtggggaacgctgtgctgtttgtatttgttcccttgggtttttttttcctgcctaatttctgtgatttccaaccaacatgaaatgactataaatggttttttaatgaaaaaagaaaaaaaaaaaaaaaaaaaaaaaaaaaaaaaaaaaaa

SEQ ID NO: 13 - protein, 671 aa, Homo sapiens
MPHAFKPGDLVFAKMKGYPHWPARIDDIADGAVKPPPNKYPIFFFGTHETAFLGPKDLFPYDKCKDKYGKPNKRKGFNEGLWEIQNNPHASYSAPPPVSSSDSEAPEANPADGSDADEDDEDRGVMAVTAVTATAASDRMESDSDSDKSSDNSGLKRKTPALKMSVSKRARKASSDLDQASVSPSEEENSESSSESEKTSDQDFTPEKKAAVRAPRRGPLGGRKKKKAPSASDSDSKADSDGAKPEPVAMARSASSSSSSSSSSDSDVSVKKPPRGRKPAEKPLPKPRGRKPKPERPPSSSSSDSDSDEVDRISEWKRRDEARRRELEARRRREQEEELRRLREQEKEEKERRRERADRGEAERGSGGSSGDELREDDEPVKKRGRKGRGRGPPSSSDSEPEAELEREAKKSAKKPQSSSTEPARKPGQKEKRVRPEEKQQAKPVKVERTRKRSEGFSMDRKVEKKKEPSVEEKLQKLHSEIKFALKVDSPDVKRCLNALEELGTLQVTSQILQKNTDVVATLKKIRRYKANKDVMEKAAEVYTRLKSRVLGPKIEAVQKVNKAGMEKEKAEEKLAGEELAGEEAPQEKAEDKPSTDLSAPVNGEATSQKGESAEDKEHEEGRDSEEGPRCGSSEDLHDSVREGPDLDRPGSDRQERERARGDSEALDEES

SEQ ID NO: 14 - protein, 669 aa, Mus musculus
MPHAFKPGDLVFAKMKGYPHWPARIDDIADGAVKPPPNKYPIFFFGTHETAFLGPKDLFPYDKCKDKYGKPNKRKGFNEGLWEIQNNPHASYSAPPPVSSSDSEAPEADLGCGSDVDKDKESRRVMTVTAVTTTATSDRMESDSDSDKSSDHSGLKRKTPVLKVSVSKRARRASSDLDQASVSPSEEDSESPSESEKTSDQDFTPEKKTAARPPRRGPLGGRKKKKVPSASDSDSKADSDGAKEEPVVTAQPSPSSSSSSSSSSSSDSDVSVKKPPRGRKPAEKPPPKPRGRRPKPERPPSTSSSDSDSDSGEVDRISEWKRRDEERRRELEARRRREQEEELRRLREQEREEKERRKERAERGGSSGEELEDEEPVKKRSRKARGRGTPSSSDSEPEGELGKEGKKLAKKSQLPGSESARKPGQKEKRGRPDEKPRARPVKVERTRKRSEGLSLERKGEKKKEPSVEERLQKLHSEIKFALKVDNPDVRKCLSALEELGTLQVTSQILQKNTDVVATLKKIRRYKANKDVMAKAAEVYTRLKSRVLGPKVEALQKVNKAGAEKERADNEKLEEQPGEQAPRELAEDEPSTDRSAPVNGEATSQKGENMEDRAQEDGQDSEDGPRGGSSEELHDSPRDNSDPAKPGNERQDHERTRLASESANDDNEDS

SEQ ID NO: 15 - protein, 226 aa, Pan troglodytes
MEEKPPARVGRLPRQPPPPPRRPAPAPGFASRCRRRRRRSRYRRCSRFPRPGPLAVSMPHAFKPGDLVFAKMKGYPHWPARIDDIADGAVKPPPNKYPIFFFGTHETAFLGPKDLFPYDKCKDKYGKPNKRKGFNEGLWEIQNNPHASYSAPPPVSSSDSEAPEANPAGGSDADEDDEDRGVMAVTAVTATAASDRMESDSDSDKSSDNSGLKRKTPALKKQRTRF

SEQ ID NO: 16 - protein, 649 aa, Canis familiaris
MPNWKIDDIADGAVKPPPNKYPIFFFGTHETAFLGPKDLFPYDKCKDKYGKPNKRKGFNEGLWEIQNNPHASYSAPLPVSSSDSEAPEADPAGGSEDDEVRGVMAVTAVTATAASDRMESDSDSDKSSDNSGLKRKAAALKMSVSKRTRKASSDLDQASVSPSEEENSESSSESEKTSDQDFTPEKKAVVRAPRRGPLAGRKKKKAPSASDSDSKAESDGAKVEPVAVARSAPSSSSSSSSSSSSDSDVSVKKPPRGRKPAEKPPPKPRGRKPKPERPPTSSSSDSDSDEVDRISEWKRRDEERRRELEARRRREQEEELRRLREQEKEEKERRRERERGEAPASGGSSGDELGDEDEPVRKRGRKGRGRGPQSSSDSGPEAELQREAKKTARQSTEPTRRPSQKEKRGRPEERPRARPAKVERTRKRSEGFPPDRKVEKKKEPSVEEKLQKLHSEIKFALKVDNPDVKRCLNALEELGTLQVTSQILQKNTDVVATLKKIRRYKANKEVMEKAAEVYTRLKSRVLGPKIEAIQKATRAGVEKERAEVEKAEEALAGEEAPMERVEDEANADLSAPVNGEATSQKGESMEDKEQEGGQNSEEGLGCGSSEEPLHNDNAREGSELDAPGKERQERARVRMDSESLDEEDS

SEQ ID NO: 17 - protein, 630 aa, Rattus norvegicus
MPHAFKPGDLVFAKMKGYPHWPARIDDIADGAVKPPPNKYPIFFFGTHETAFLGPKDLFPYDKCKDKYGKPNKRKGFNEGLWEIQNNPHASYSAPLPVSSSDSEAPEADLGGGSDADKEKEARRVMTVTAVTTTATSGRTESDSDSDKNSDHSGLKRKTPVLKMSVSKRARKASSDLDQASVSPSEEDSESPSESEKTSDQDFTPEKKTIARAPRRAPLGGRKKKKVPSASDSDSRADSDGAKEEPVVTAQPSPSSASSSSSSSASDSDVSIKKPPRGRKPAEKPPPKPRGRRSKPERPPSTSSSDSDSDSGEVDRISEWKRRDEERRRELEARRRREQEEELRRLREQEREEKERRKERAERGGSSGEELEDEEPVKKRSRKARGRGTPSSSDSEPEGELGKEGKKLAKKSQLQGSESARKPGQKEKRGRPDEKPRARPVKVERTRKRSEGLSLDRKGEKKKEPSVEERLQKLHSEIKFALKVDNPDVRRCLSALEELGTLQVTSQILQKNTDVVATLKKIRRYKANKDVMAKAAEVYTRLKSRVLGPKVEALQKVNKAGAEKERADGEKVEEQPGEQAPRELAEDEPSTDRSAPVNGEAASQKGENTEDGAQEDGQDLEDGPRGGSSE

SEQ ID NO: 18 - DNA, 1973 bp, Homo sapiens
agccgccgcctcgccgcttcccctcgtcggagcggccgctcgtccgcccggcttgaggcccgcggggagcgcggcgcaattcgtcggcccgcgggggggcggcctcccggcatcttcgcggcgaccaaggactaccaggaaggggagcggctgggatggcgcgtccgcggccccgcgagtacaaagcgggcgacctggtcttcgccaagatgaagggctacccgcactggccggcccggattgatgaactcccagagggcgctgtgaagcctccagcaaacaagtatcctatcttcttttttggcacccatgaaactgcatttctaggtcccaaagacctttttccatataaggagtacaaagacaagtttggaaagtcaaacaaacggaaaggatttaacgaaggattgtgggaaatagaaaataacccaggagtaaagtttactggctaccaggcaattcagcaacagagctcttcagaaactgagggagaaggtggaaatactgcagatgcaagcagtgaggaagaaggtgatagagtagaagaagatggaaaaggcaaaagaaagaatgaaaaagcaggctcaaaacggaaaaagtcatatacttcaaagaaatcctctaaacagtcccggaaatctccaggagatgaagatgacaaagactgcaaagaagaggaaaacaaaagcagctctgagggtggagatgcgggcaacgacacaagaaacacaacttcagacttgcagaaaaccagtgaagggacctaactaccataatgaatgctgcatattaagagaaaccacaagaaggttatatgtttggttgtctaatattcttggatttgatatgaaccaacacatagtccttgttgtcattgacagaaccccagtttgtatgtacattattcatattcctctctgttgtgtttcggggggaaaagacattttagccttttttaaaagttactgatttaatttcatgttatttggttgcatgaagttgcccttaaccactaaggattatcaagatttttgcgcagacttatacatgtctaggatccttttatcaaggcagttatgatcatcgttttcctgccttgaccccaccatcatcaaacactcagttaaatataaattaacattttttagatgaccactcaacataatgcttaagaatggaatttcctctctgtgacagaacccaggaattaattcctaaatacataacgttggtatattgaagacgaaattaaaattgtccttcagttttgaggccatgtgtaaagtttaaccatattgtaaaatatctattccgtattagaaatagctagttgacagcttatacttctcaaaattcatattgttatgtacacaaactaagtttctatatgtgaagttagtgagtctttttgtgttactccaaaataaaggcaatgatttatttttttcccagtgccaatacaattttgagctaagcactcaaggtggatactttacattttaaagctggaatcagcaacagccctatgggaaaccagacaaagcattgacttttaaatgtagacttttaaaataaactgttttcttttggaactacaattagaatagttaatattcatccttaaaccattattatgtgtacattattgttgctattgtgataatagagaattttatttatttttatgccagcttatattgtgagaacacatttagtcagtttgggttttatcaatcctgttaatgcttgtccttggaacatctttcgcgtattcacggtttgtagttgaaaagtttactgtaaaaaaatcaaaaacaaaaaaatgtattgtttttacagaataaatttattgggatgtgtactgggagtaagatttgaggttgtaagcaaactaagttagtgtaatttggcttcatatatgtaacgtgaggtattaatgtaattcatatattaaagcaaaaattgttcgcagc

SEQ ID NO: 19 - protein, 203 aa, Homo sapiens
MARPRPREYKAGDLVFAKMKGYPHWPARIDELPEGAVKPPANKYPIFFFGTHETAFLGPKDLFPYKEYKDKFGKSNKRKGFNEGLWEIENNPGVKFTGYQAIQQQSSSETEGEGGNTADASSEEEGDRVEEDGKGKRKNEKAGSKRKKSYTSKKSSKQSRKSPGDEDDKDCKEEENKSSSEGGDAGNDTRNTTSDLQKTSEGT

SEQ ID NO: 20 - protein, 207 aa, Canis familiaris
MVINLRDLSGAELGELGEKLNTGLEEITNKIKIDELPEGAVKPPANKYPIFFFGTHETAFLGPKDLFPYKEYKDKFGKSNKRKGFNEGLWEIENNPGVKFTGYQAIQQQSSSETEGEGGNTADASSEEEGDRVEEDGKGKRKNEKAGSKRKKSYTSKKSSKQSRKSPGDEDDKDCKEEENKSSSEGGDAGNDTRNTTSDLQKTSEGT

SEQ ID NO: 21 - protein, 202 aa, Mus musculus
MARPRPREYKAGDLVFAKMKGYPHWPARIDELPEGAVKPPANKYPIFFFGTHETAFLGPKDLFPYKEYKDKFGKSNKRKGFNEGLWEIENNPGVKFTGYQTIQQQSSSETEGEGGNTADASSEEEGDRVEDGKGKRKNEKGGSKRKKSYTSKKSSKQSRKSPGDEDDKDCKEEENKSSSEGGDAGNDTRNTTADLQKAGEGT

SEQ ID NO: 22 - protein, 202 aa, Rattus norvegicus
MARPRPREYKAGDLVFAKMKGYPHWPARIDELPEGAVKPPANKYPIFFFGTHETAFLGPKDLFPYKEYKDKFGKSNKRKGFNEGLWEIENNPGVKFTGYQTIQQQSSSETEGEGGNTADASSEEEGDRVEDGKGKRKNEKGGSKRKKSYTSKKSSKQSRKSPGDEDDKDCKEEENKSSSEGGDAGNDTRNTTSDLQKAGEGT

SEQ ID NO: 23 - protein, 539 aa, Gallus gallus
METTLNVWILSKKAGLPVTQWQELHLSKTVCQLHRSGRAIAEGGAVVKTAVCGQLLSLQRPKAVYSCFSAAAPTANKTGPGGGRLAVPRGPGGCDLPCAQAGTLLQQVSRAVPFFVSQVFVSTPCKLQCAKVSVRTSGTSFSSRLDREGQRPGQGEPNGLLGSPSAIFSSAQKSHPDRLEFVLLTNSKSAAQGVWAALLSLGAIAWTWDALCCVRQRCLCLDCCSNGKQSSAEQNTQEGLQPPLLPHQLFQGADPPASSCRGCFVDGEPCCSRGAAQCWGCGAAAAETVNFELDKMEGQGNGQKQAVGLQGKESAGASSLVCVVCSTPGATITTSVREFLSQLGRALPQDPTVGSNAVSISVSQIDELPEGAVKPPANKYPIFFFGTHETAFLGPKDLFPYKEYKDKFGKSNKRKGFNEGLWEIENNPGVKFTGYQAIQQQSSSETEGEGGNTADASSEEEGDRVEEDGKGKRKNEKAGSKRKKSYTSKKSSKQSRKSPGDEDDKDCKEEENKSSSDAGDAGNDTRNTSSDLQKTSEGT

SEQ ID NO: 24 - protein, 235 aa, Bos taurus
MSRFYRRKYKCGDLVFAKLKGYAHWPARIEQTAEANRYQVFFFGTHETAFLGPRHLFPYEESKEKFGKPNKRRGFSEGLWEIENNPTVQASDYQCALEKSCPEEPEPEVAEGGEDPKSHTNGGDDDDQGKLGVDLPAEEENKKETLKRTAEDPPEDIPKRPKEADPEEGEERKEAAAVAEEAEDARPLLVEVENDPAASVLGLAWGLPVMEQEPEEESAEREAQAPGVGGGHESQ

SEQ ID NO: 25 - DNA, 2346 bp, Homo sapiens
gacccccggtctcgcccccgaaacatgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagagggaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcagcagaataaagatgaaggaaagaagccagaagttaagaaagtggagaagaagcgagaaacatcaatggattctcgacttcaaaggatacatgctgagattaaaaattcactcaaaattgataatcttgatgtgaacagatgcattgaggccttggatgaacttgcttcacttcaggtcacaatgcaacaagctcagaaacacacagagatgattactacactgaaaaaaatacggcgattcaaagttagtcaggtaatcatggaaaagtctacaatgttgtttaacaagtttaagaacatgttcttggttggtgaaggagattccgtgatcacccaagtgctgaataaatctcttgctgaacaaagacagcatgaggaagcgaataaaaccaaagatcaagggaagaaagggccaaacaaaaagctagagaaggaacaaacagggtcaaagactctaaatggaggatctgatgctcaagatggtaatcagccacaacataacggggagagcaatgaagacagcaaagacaaccatgaagccagcacgaagaaaaagccatccagtgaagagagagagactgaaatatctctgaaggattctacactagataactaggttgacatacctggaatatagagaacacttgagaagtttgtaatggttttcatttgaaatagactgctgaaagttttaaatttttataagcataggtttgatgttgaaaacttgttttgagggagaaaatccctttgttttaaagtaaagtaaacattatcgctaagtgtacttgtgcagtattaacagctacattatacagtaaatgtgggatggaatccatttaggaaatgttaaactgcttttccagacatggttgtagcatattttcaattagtgtgtgtatgttaatgtgtaattgatagtagaacaaagttacatttttaaaactgctacttgtataaaccttgcctcttttcccaaatactgtgggttttgtgcatagtttttacaaaccttggatttaccagactgtcttttcactgtttgtgggttttgtagaagttacacatttttatggtagataaaatgttacttctatacaagtactcactccctttttatcaaaagttaattttaatctcacagtctacattgtgctacattatccagcttctttggaacaatgtgtgctctgtatggttttttttggtatgacaactaattaagcaactgacattgaactgagaattctacaaactataaaacattaatttttgaaggtaatttagttttgtggctgggcattcagtgaagtcttaggacttctttgcagacaactgactgggt

SEQ ID NO: 26 - protein, 530 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEGKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLFNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 27 - protein, 528 aa, Mus musculus
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQASTKQSNASSDVEVEEKETNVSKEDTDQEEKASNEDVTKAVDITTPKAARRGRKRKAEKQVDTEEAGMVTAATASNVKASPKRGRPAATEVKIPKPRGRPKVVKQPCPSDGDMVIDEDKSKKKGPEEKQPKKQLKKEEEGQKEEEKPRKEPDKKEGKKEVESKRKNLAKPGVTSTSDSEDEDDQEGEKKRKGGRNFQAAHRRNMLKGQHEKEAGDRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEPTGTKSLNGGSDAQESNHPQHNGDSNEDGKDSREASSKTKPPGEEREAEISLKESTLDN

SEQ ID NO: 28 - protein, 528 aa, Rattus norvegicus
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQVSTKQSNASSDVEAEEKETSVSKEDTDQEEKASNEDVTKAADITTPKAARRGRKRKAEKQVDTEEAGVVTAATASNVKASPKRGRPAATEVKIPKPRGRPKVVKQPCPSESDMVIDEDKSKKKGPEEKPPKKQLKKEEEGQKEEEKPRKEPDKKEGKKEVESKRKNLAKPGVTSTSDSEEDDDQEGEKKRKGGRHFQAAHRRNMLKGQHEKEAADRKRKQEEQMETEQQTKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGTKSLNGGSDAQESNHPQHNGDSAEESKDSREAGSKTKTPGEEREAEVSLKESTLDN

SEQ ID NO: 29 - protein, 483 aa, Bos taurus
HETAFLGPKDIFPYSENKEKYGKPNKRKGFNEGLWEIDNNPKVKFSSQQASAKQSNASSDVEVEEKETSVSKEDTDPEEKASNEDVTKAIDITTPKAARRGRKRKAEKQVETEEAGVVTTATASANLKVSPKRGRPAATEVKIPKPRGRPKMVKQPCPSESDMITEEDKSKKKGQEEKQPKKQLKKDEEGQKEEEKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGSKTLNGGSDAQDSNQPQHNGDSNEESKDNHEASSKKKPSSEERETEISLKDSTLDN

SEQ ID NO: 30 - protein, 579 aa, gallus gallus
MSRDFKPGDLIFAKMKGYPHWPARVDEVPDGAVKPPTNKMPIFFFGTHETAFLGPKDIFPYSENKDKYGKPNKRKGFNEGLWEIDNNPKVKFSHQPSHPAVNTSIKETIQESSPEAAEGSEEKSGAKRRKPSIPKLPPKGDNNSPAEAEAEEKEMHSTKEDEEPSEKNSKEGVVKTNDASIPKVARRGRKRKAEKQAESEEAAVVATAAVVAAAAAPVTVSPKVSPKRGRPAVSEVKVPKPRGRPKLVKPSCLSESDPVNEEEKAKKKGPDEKPKKQGKKDEEGQKEEEKPKKEYDKKDGKKEAEPKRKNAAKLGSASASDSEDEGGEEEGDKKKKGGRSFQSTHRRNIMRGQHEKEVTERKRKQEEQGESELQNKEEGKKTEVKKMEKKRETSMDSRLQRIHAEIKNSLKIDNLDVNRCIEALDELASLQVSMQQAQKHTEMILTLKKIRKFKVSQVIMEKSTMLYNKFKTMFLVGEGDSVLSQVLNKSLAEQKQHEEANKTKEQWKKGTNKKNEKEKDQTGSKIVNGGSETQDTNQSQHNGENAEEKDKLEVASKKKTCGEESELEKPAKESAFENK

SEQ ID NO: 31 - DNA, 342 bp, Artificial
  C1 heavy chain nt
gaggtgcagcttgttgagtctggtggaagattggtgcagcctaaagggtcattgaaactctcatgtgcagcctctggattcaccttcaatacctatgccatgtactggatccgccaggctccaggaaagggtttggaatgggttgctcgcataagaagtaaaagttataattatgcaacatattatgccgattcagtgaaagacagattcaccatctccagagatgattcacaaagcatgctctatctgcaaatgaacaacctgaaaactgcggacacagccatgtattactgtgtgagtgagggattttggggtcaaggaacctcagtcaccgtctcctca

SEQ ID NO: 32 - protein, 114 aa, Artificial
  C1 heavy chain aa
EVQLVESGGRLVQPKGSLKLSCAASGFTFNTYAMYWIRQAPGKGLEWVARIRSKSYNYATYYADSVKDRFTISRDDSQSMLYLQMNNLKTADTAMYYCVSEGFWGQGTSVTVSS

SEQ ID NO: 33 - DNA, 336 bp, Artificial
  C1 light chain nt
gatgttttgatgacccaaactccactctccctgcctgtcagtcttggagatcaagcctccatctcttgcagatctagtcagagcattgtacatagtagtggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 34 - protein, 112 aa, Artificial
  C1 light chain aa
DVLMTQTPLSLPVSLGDQASISCRSSQSIVHSSGNTYLEWYLQKPGQSPKLLIYKVSNRFSGVPDRFSGSGSGTDFTLKISRVEAEDLGVYYCFQGSHVPWTFGGGTKLEIK

SEQ ID NO: 35 - DNA, 360 bp, artificial
  H3 heavy chain nt
cagattactctgaaacagtctggccctgggatagtgcagccatcccagcccgtcagacttacttgcactttctctgggttttcactgagcacttctggtataggtgtagcctggattcgtcagccctcagggaagggtctggagtggctggcaaccatttggtgggatgatgataaccgctacaacccatctctaaagagcaggctcgcagtctccaaagacacctccaacaaccaagcattcctgaatatcatcactgtggaaactgcagatactgccatatactactgtgctcagatctatgattacgccgtgggttttgcttattggggccaagggactctggtcactgtctctgca

SEQ ID NO: 36 - protein, 120 aa, artificial
  H3 heavy chain aa
QITLKQSGPGIVQPSQPVRLTCTFSGFSLSTSGIGVAWIRQPSGKGLEWLATIWWDDDNRYNPSLKSRLAVSKDTSNNQAFLNIITVETADTAIYYCAQIYDYAVGFAYWGQGTLVTVSA

SEQ ID NO: 37 - DNA, 318 bp, artificial
  H3 light chain nt
gatatccagatgacacagactacatcctccctgtctgcctctctgggagacagagtcaccatcagttgcagggcaagtcaggatattagcaattatttaaactggtatcagcagaaaccagatggaactgttaaactcctgatctactacacatcaagattacactcaggagtcccatcaaggttcagtggcagtgggtctgggacagattattctctcaccatcagcaacctggaacctgacgatattgccacttactattgtcaacaatatggtaagttgtggacgttcggtggaggcaccaagctggaaatcaaa

SEQ ID NO: 38 - protein, 106 aa, artificial
  H3 light chain aa
DIQMTQTTSSLSASLGDRVTISCRASQDISNYLNWYQQKPDGTVKLLIYYTSRLHSGVPSRFSGSGSGTDYSLTISNLEPDDIATYYCQQYGKLWTFGGGTKLEIK

SEQ ID NO: 39 - DNA, 399 bp, artificial
  C1 heavy chain nt
atggtgatggggctgaagtgggttttctttgctgttttttatcaaggtgtgcattgtgaggtgcagcttgttgagtctggtggaagattggtgcagcctaaagggtcattgaaactctcatgtgcagcctctggattcaccttcaatacctatgccatgtactggatccgccaggctccaggaaagggtttggaatgggttgctcgcataagaagtaaaagttataattatgcaacatattatgccgattcagtgaaagacagattcaccatctccagagatgattcacaaagcatgctctatctgcaaatgaacaacctgaaaactgcggacacagccatgtattactgtgtgagtgagggattttggggtcaaggaacctcagtcaccgtctcctca

SEQ ID NO: 40 - protein, 133 aa, artificial
  C1 heavy chain aa
MVMGLKWVFFAVFYQGVHCEVQLVESGGRLVQPKGSLKLSCAASGFTFNTYAMYWIRQAPGKGLEWVARIRSKSYNYATYYADSVKDRFTISRDDSQSMLYLQMNNLKTADTAMYYCVSEGFWGQGTSVTVSS

SEQ ID NO: 41 - DNA, 393 bp, Artificial
  C1 light chain nt
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtagtggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 42 - protein, 131 aa, Artificial
  C1 light chain aa
MKLPVRLLVLMFWIPASSSDVLMTQTPLSLPVSLGDQASISCRSSQSIVHSSGNTYLEWYLQKPGQSPKLLIYKVSNRFSGVPDRFSGSGSGTDFTLKISRVEAEDLGVYYCFQGSHVPWTFGGGTKLEIK

SEQ ID NO: 43 - DNA, 417 bp, artificial
  H3 heavy chain nt
atggacaggcttatgtcttcattcctgctgctggttgtccctatatatgttctgtcccagattactctgaaacagtctggccctgggatagtgcagccatcccagcccgtcagacttacttgcactttctctgggttttcactgagcacttctggtataggtgtagcctggattcgtcagccctcagggaagggtctggagtggctggcaaccatttggtgggatgatgataaccgctacaacccatctctaaagagcaggctcgcagtctccaaagacacctccaacaaccaagcattcctgaatatcatcactgtggaaactgcagatactgccatatactactgtgctcagatctatgattacgccgtgggttttgcttattggggccaagggactctggtcactgtctctgca

SEQ ID NO: 44 - protein, 139 aa, artificial
  H3 heavy chain aa
MDRLMSSFLLLVVPIYVLSQITLKQSGPGIVQPSQPVRLTCTFSGFSLSTSGIGVAWIRQPSGKGLEWLATIWWDDDNRYNPSLKSRLAVSKDTSNNQAFLNIITVETADTAIYYCAQIYDYAVGFAYWGQGTLVTVSA

SEQ ID NO: 45 - DNA, 378 bp, artificial
  H3 light chain nt
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggatattagcaattatttaaactggtatcagcagaaaccagatggaactgttaaactcctgatctactacacatcaagattacactcaggagtcccatcaaggttcagtggcagtgggtctgggacagattattctctcaccatcagcaacctggaacctgacgatattgccacttactattgtcaacaatatggtaagttgtggacgttcggtggaggcaccaagctggaaatcaaa

SEQ ID NO: 46 - protein, 126 aa, artificial
  H3 light chain aa
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEPDDIATYYCQQYGKLWTFGGGTKLEIK

SEQ ID NO: 47 - RNA, 21 nt, Artificial
  siRNA-1
aaccggcagaaggaguacaaa

SEQ ID NO: 48 - RNA, 21 nt, Artificial
  siRNA-2
aaaucaacagccaacaaauac

(19) United States
(12) Patent Application Publication
(10) Pub. No.: US 2007/0243191 A1
(43) Pub. Date: Oct. 18, 2007

(54) ANTI-HYPERPROLIFERATIVE THERAPIES TARGETING HDGF

(75) Inventors: Li Mao, Houston, TX (US); Hening Ren, Houston, TX (US); Jun Zhang, Shenyang (CN)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/611,554

(22) Filed: Dec. 15, 2006

Related U.S. Application Data
(60) Provisional application No. 60/753,600, filed on Dec. 23, 2005.
(60) Provisional application No. 60/810,062, filed on May 31, 2006.

(51) Int. Cl. A61K 39/395; A61K 31/70; A61P 35/00; C07H 21/02; C07K 16/18; C12Q 1/00

(52) U.S. Cl. 424/138.1; 424/130.1; 435/7.23; 514/44; 530/388.1; 536/23.1

(57) ABSTRACT

The present invention provides methods of cancer therapy or diagnosis involving targeting hepatoma-derived growth factor (HDGF). In certain embodiments, an antibody and/or siRNA may be used to inhibit HDGF, optionally coupled to or combined with other cancer therapies.

This application claims priority under 35 U.S.C. 119(e) to U.S. Provisional Patent Application 60/753,600 filed on Dec. 23, 2005, and U.S. Provisional Patent Application 60/810,062 filed on May 31, 2006, all of which are hereby incorporated by reference in their entirety.

The government owns rights in the present invention pursuant to Department of Defense grant DAMD17-01-1-01689-1 and National Cancer Institute grants PO1 CA106451, PO1 CA91844, and U01 CA86390.

### BACKGROUND OF THE INVENTION

1. Field of the Invention

The present invention relates generally to the fields of molecular biology and oncology. More particularly, it concerns methods of hyperproliferative cell, and particularly cancer, therapy involving the suppression of HDGF, e.g., via an antibody and/or siRNA, alone or in combination with secondary therapeutics.

2. Description of the Related Art

Lung cancer is the leading cause of cancer-related death in the United States in both men and women (Parkin et al., 1999). In 2006, more than 174,470 new cases of lung cancer are estimated in the United States alone, and 162,460 patients with lung cancer will die of the disease (Jemal et al., 2006). The five-year survival rate for lung cancer is about 15% in the United States (id.), while poorer survival rates are observed in Europe (8%) and developing countries (Jemal et al., 2002). Although the 5-year survival rate can be as high as 50% for patients diagnosed at localized stage and treated by surgery (Nesbitt et al., 1995), less than 20% of lung cancer patients are diagnosed at this earlier stage. Recent studies demonstrated that the overall survival may be farther improved in this population with the addition of adjuvant chemotherapy (Arriagada et al., 2004). Therefore, the identification of reliable prognostic factors for disease recurrence and death could have significant clinical implications because patients in the high-risk groups, for example, would be appropriate candidates for novel postoperative adjuvant therapies whereas these therapies may not be necessary for the low-risk patients.

Unfortunately, current clinical tools cannot provide guidance regarding who is likely to be cured by surgery alone and who is destined to experience disease recurrence or metastasis. Because molecular alterations are what underlie the biologic diversities of lung cancers, they may serve as biomarkers for risk assessment and possibly therapeutic targets. Such strategy can also apply to patients with advanced stages of lung cancer. For the later groups of patients (>80% of all lung cancer patients), the median survival has only been improved by a few months over past two decades despite extensive efforts in the new drug development and the use of combination chemotherapeutic regimens (Crino et al., 2002). Clearly, better understanding biology of this disease and development of novel therapeutic strategies are urgently needed.

With the advances in our understanding of the pathophysiology during tumorigenesis, we now know that many oncogenes and tumor suppressor genes are altered in lung cancers (Mao et al., 2001). These alterations are the basis for malignant transformation, immortalization, increased proliferation, anti-apoptosis, invasion, and metastasis. Despite the similarities in gross morphology and histopathology among each subgroup of lung cancers, differences in the patterns of molecular alterations in tumor cells and their microenvironment dictate tumors' biological behaviors and consequently, impact fate of the patients.

In fact, molecular classification has been successfully integrated into the clinical management of patients with breast cancer. The expression level of estrogen receptor (ER) in tumor cells is a useful indicator in predicting clinical outcome of patients with breast cancer (Clark et al., 1988). The understanding of the role of ER in patients with breast cancer led to the development of estrogen antagonists. Such antagonists, such as tamoxifen, have been successfully used to treat patients with ER positive breast cancer (Barker, 2003) and therefore ER status becomes a useful marker for patient selection.

Another example is the discovery of HER2 gene amplification and its association with poor clinical outcome in patients with breast cancer (Volpi et al. 2003). The discovery led to the development of trastuzumab (Herceptin®), an anti-Her-2 antibody, to treat patients with HER2 amplified breast cancer (Slamon et al., 2001).

More recently, EGFR tyrosine kinase inhibitors, such as Tarceva® or Erbitux®, have proven to be effective in treating patients whose NSCLC tumors had EGFR mutations and/or gene amplification (Paez et al., 2004). The development of EGFR inhibitors was based on earlier findings that EGFR is frequently overexpressed in cancers including lung cancer and the overexpression correlated with poor clinical outcomes (Pastorino et al., 1997). These successful examples demonstrate the importance of identifying key molecular alterations in cancer classification and in the development of targeted therapies.

HDGF is a heparin-binding growth factor originally purified from media conditioned by a human hepatoma-derived cell line, Huh-7 (Nakamura et al., 1989). HDGF fits the definition of a growth factor because the exogenous HDGF is mitogenic to fibroblasts, HuH-7 cells (id.), aortic endothelial cells (Everett, 2001), and vascular smooth muscle cells (Oliver et al., 2001). The HDGF family of growth factors (variously referred to collectively herein as “HDGF”) represent a new family of growth factors in that the nearest sequence homology (32%) is to high mobility group-1 (HMG-1), a DNA-binding protein (Nakamura et al., 1994). Indeed, HDGF family members lack many of the specific features characteristic of an HMG protein, such as an “HMG box,” which is responsible for DNA binding of these proteins. Instead, HDGF carries a PWWP domain which has been shown to be able to bind DNA directly in proteins such as DNMT3B, a DNA methyltransferase (Qiu et al., 2002). The PWWP domain is frequently found in proteins associated with chromatin (Slater et al., 2003) and is proposed to have a protein-protein binding capability (Stec et al., 2000). The HDGF-related proteins have been described so far including mouse HRP-1, mouse HRP-2, human and mouse HRP-3, bovine HRP-4, and human LEDGF (lens epithelium-derived growth factor) (Izumoto et al., 1997; Ikegame et al., 1999; Abouzied et al., 2004; Dietz et al., 2002; Singh et al., 2002).

The folding structure of HDGF has been reported in recent studies (Sue et al., 2004). A heparin binding pocket, the characteristic feature of growth factors, is identified at the N-terminal of the protein (amino acids 19-80) and shows a strong binding activity to heparin (id.). More recently, a surface receptor binding motif was identified adjacent to the heparin binding pocket (amino acids 81-100) and the binding of HDGF to the receptor is important for stimulating growth of fibroblast cells without nuclear internalization (Abouzied et al., 2005). Together these data support the notion that HDGF has two distinct domains with mitogenic capability, one through receptor binding and the other through transcription regulation once the protein is translocated into nuclear. Given the fact that HDGF can be released into extracellular space and re-entered into cells, it may function not only in cells producing the protein but also function in autocrine and paracrine manners.

Over-expression of HDGF was first found in primary esophageal cancers, associated with radiation sensitivity (Matsuyama et al., 2001). Overexpression of HDGF was subsequently reported in CML (Bruchova et al., 2002), liver cancer (Hu et al., 2003), colorectal cancer (Lepourcelet et al., 2005), melanoma (Bernard et al., 2003), and gastric cancer (Yamamoto et al., 2006). In these studies, patients whose tumors expressed higher levels of HDGF had significantly poorer survivals than those whose tumors expressed lower levels of HDGF in patients with liver cancers, colorectal cancers, and gastric cancers. In melanoma, high level of HDGF correlated with advanced stage of tumors, invasion, and metastasis (Bernard et al., 2003). The present inventors' laboratory reported that overexpression of HDGF in NSCLC, and a strong correlation between HDGF expression levels in the primary tumors and patients' clinical outcomes (Ren et al., 2004).

Little has been previously known about HDOF as a potential therapeutic target in cancer. Kishima et al. reported the use of antisense oligonucleotides or adenoviral vectors down-regulate HDGF gene expression in hepatoma cells in vitro (Kishima et al., 2002). While these authors observed an inhibition of cell proliferation in an in vitro hepatocellular carcinoma model, they relied on in vitro observations and were carried out only in hepatocellular carcinoma culture cells.

From the foregoing it is apparent that a substantial need exists to identify novel molecular, tumor-specific targets and to develop new therapeutics that will permit oncologists to selectively treat cancer by means of such targets. The present inventors present herein their discovery that HDGF, and its related family members and receptors, is a very important target for clinical anti-hyperproliferative therapy, particularly, for example, using small inhibitory nucleic acid or antibody molecules.

### SUMMARY OF THE INVENTION

The present invention overcomes deficiencies in the prior art by identifying that HDGF may be used as a target for hyperproliferative cell, and in particular cancer, therapies, and identifying that HDGF may be employed as a diagnostic or prognostic marker; furthermore, the present invention provides therapies that selectively target and neutralize HDGF function. In certain embodiments, a small inhibitory nucleic acid (“siNA”) or an antibody (e.g., a monoclonal antibody, an antibody fragment or single chain antibody, or a humanized, fully human and/or recombinant version of the foregoing) which selectively affects HDGF (i.e., binds, reduces the function of and/or reduces the expression of HDGF) may be used to treat such disorders.

Accordingly, in certain embodiments the present invention is directed to a method of reducing the growth of or inducing apoptosis in hyperproliferative cells comprising administering to said cells an amount of an HDGF-targeting agent that down-regulates HDGF action that is effective to reduce the growth of or induce apoptosis in said cells. The method may be performed in vitro, for example as a means of assessing the ability of the agent to effectively target a particular cell population or cell type or to assess the agent's ability to target a particular HDGF or homologue or family member thereof, or the method may be a therapeutic or diagnostic method performed in vivo wherein the hyperproliferative cells are in a subject and the HDGF-targeting agent is administered to the subject.

In some embodiments the method may be performed on an animal such as a laboratory animal wherein the subject is a mouse or rat, or on a human patient, such as a patient suffering from a hyperplastic disorder such as cancer. In such embodiments, the method may be experimental or diagnostic in nature and performed, for example, to determine the ability of the agent to effectively target or down-regulate HDGF in a particular cell population or cell type, or to assess the ability of the agent to target and down-regulate a particular HDGF or homologue or family member thereof. Of course, therapeutic applications in the treatment of hyperproliferative cells in an animal is considered to be within the scope of the invention as well.

In certain applications, the method will result in a reduction in the growth of the treated cells which, in the case of a cancer treatment will typically manifest itself in a reduction in overall tumor size or mass. Alternatively or additionally, particularly in the context of a solid tumor mass, the method may result in a reduction in the invasiveness of a tumor or tumor mass, and/or effect anti-angiogenesis wherein blood flow to the tumor mass is reduced, thus, in effect, “starving” the tumor. In still further embodiments, particularly when the agent is co-administered with a second therapeutic agent (such as a chemotherapeutic or molecular targeting agent), the treatment will induce apoptosis in cells of the cell population being treated.

As described in more detail hereinbelow, the inventors have discovered that those cells that overexpress HDGF are typically more sensitive to therapy with the HDGF-targeting therapeutic. Cells that overexpress HDGF may be characterized as cells that express a higher level of HDGF relative to normal cells such as normal lung cells or vascular smooth muscle cells. An exemplary cell line that expresses “normal” levels of HDGF (i.e., cells that do not overexpress HDGF) include cell line NCI-H522 cells (ATCC CRL-5801). However, in many instances a convenient, although not always the most precise, means of determining whether a cell overexpresses HDGF is by determining whether the HDGF expression by the cell is detectable by immunohistochemistry (IHC). The reason for this is that IHC detection techniques are not as sensitive a detection means as other molecular techniques (such as mRNA hybridization and PCR detection methods). It is typically the case that if the cell expresses an amount of HDGF that is detectable by IHC, then that cell is an HDGF overexpressor.

In certain preferred embodiments of the invention, the therapeutic or diagnostic agent is an siNA or an antibody that recognizes HDGF. An siNA molecule is a small inhibitory nucleic acid, that may be either an siRNA (i.e., a small inhibitory ribonucleic acid) or it may be a DNA molecule that is structured to express an siRNA when introduced into a cell. In either case, a preferred siRNA for use on the practice of the present invention will be a siRNA that designed to knock down HDGF expression, or expression of an HDGF family member or homologue. Exemplary siNAs are siRNAs that incorporate SEQ ID NO:47 or SEQ ID NO:48, or an siRNA-encoding DNA that encodes SEQ ID NO:47 or SEQ ID NO:48. A particularly preferred example of the former is referred to herein as HDGF-siRNA-1 or HDGF-siRNA-2.

In other preferred embodiments, the HDGF-targeting agent is an antibody. Particularly preferred are antibodies that recognize and bind to native HDGF and acts to neutralize the action of the bound HDGF. The antibody may be an IgG, IgM, IgA, IgD or IgE. In certain embodiments, IgG and/or IgM are preferred because they are the most common antibodies in the physiological situation and because they are most easily made in a laboratory setting. Typically preferred are monoclonal antibodies. In addition to the ease of standard preparation of therapeutically acceptable commercial amounts of monoclonal antibodies, the use of monoclonal antibodies were found by the inventors to have a somewhat unexpected benefit in that all of the anti-HDGF antibodies known to the present inventors, prior to the present invention, are not HDGF-neutralizing (see, e.g., Lepourcelet et al., 2005), for example, likely because they do not recognize, or recognize only poorly, native HDGF molecules.

In some embodiments, the HDGF-targeting agent comprises a heavy chain variable region comprising SEQ ID NO:32 and a light chain variable region comprising SEQ ID NO:34. In another embodiment, the HDGF targeting agent comprises a heavy chain variable region comprising SEQ ID NO:36 and a light chain variable region comprising SEQ ID NO:38.

Another embodiment of an HDGF-targeting agent is an isolated nucleic acid that encodes the amino acid sequences of SEQ ID NO: 32 and SEQ ID NO:34; or SEQ ID NO:36 and SEQ ID NO:38.

Exemplary monoclonal antibodies described herein include those designated C1, H3, L5-9, C4, I4, D5 or A2, with antibodies C1 or H3 being most preferred. Hybridomas expressing the more preferred of the foregoing monoclonals, H3, C1, C4 and L5-9, were deposited with the American Type Culture Collection on Dec. 14, 2006 and received designations as follows:

HDGF H3a8p17 (“H3”): ______

HDGF C1-2f (“C1”): ______

HDGF C4-3L (“C4”): ______

HDGF L5-9.6LP15 (“L5-9”): ______

The ATCC is located at 10801 University Boulevard, Manassas, Va. 20110-2209, USA, The ATCC deposits were made pursuant to the terms of the Budapest Treaty on the international recognition of the deposit of microorganisms for purposes of patent procedure.

The inventors contemplate that antibody derivatives such as Fab′, Fab, F(ab)s, DAB, Fv or scFv will find useful application in accordance with the invention. Further, it is contemplated that the most preferred antibodies will be chimeric, humanized, or human anti-HDGF antibodies, such as chimeric, humanized, or human versions of one of the foregoing listed antibodies. Accordingly, as used herein, the term “antibody” or “monoclonal antibody” is intended to include any of the foregoing, including but not limited to IgG, IgM, IgA, IgD and IgE antibodies, as well as antibody derivates including but not limited to Fab′, Fab, F(ab)s, DAB, Fv or scFv antibodies.

In certain embodiments, the present inventors contemplate that the HDGF targeted therapeutics of the present invention will recognize and/or target HDGF per se and/or will also recognize and/or target an HDGF homologue or family member. Most preferred will be those agents directed to human HDGF per se. However, agents that alternatively or additionally target other HDGF family members or homologues are within the scope of the invention. Included within the definition of HDGF, therefore, are HDGF family members such as HDGF from species other than humans (e.g., chimpanzee, canine, murine, rat, bovine, etc.), as well as other members of the HDGF family of proteins, including the HDGF-related proteins (“HRPs”). Exemplary HRPs include HRP-1, HRP-2, HRP-3, HRP-4 and p52/75 or LEDGF (lens epithelium-derived growth factor). Examples of the foregoing that have been listed on sequence databases include canineHRP-1, ratHRP-1, humanHRP-2, murineHRP-2, chimpHRP-2, canineHRP-3, murineHRP-3, ratHRP-3, chickenHRP-3, bovineHRP-4, humanP52/p75, murine P52/p75, rat P52/p75, bovine P52/p75 and chicken P52/p75.

In certain embodiments, the invention contemplates assessing the HDGF expression of the cell, such as in a tumor or suspected tumor mass, either before or after administration of the agent. For reasons discussed above, such expression is preferably assessed by means of immunohistochemistry. However, the use of molecular techniques such as but not limited to nucleic acid hybridization, PCR, ELISA, gel electrophoresis, MALDI, nucleic acid chips and arrays, protein arrays, flowcytometry, biosensors, nanoparticles or the like, is considered to be within the scope of the invention.

In still other embodiments, HDGF antibody is linked to a diagnostic or therapeutic molecule. In such embodiment, the diagnostic molecule may be a reporter molecule such as a radioligand or a fluorescent label. In therapeutic embodiments, the antibody may be an “immunotoxin” wherein the antibody is linked (e.g., recombinantly fused or chemically conjugated) to a therapeutic molecule, including but not limited to a toxin, an apoptotic molecule, an antitumor agent, a therapeutic enzyme or a cytokine. It should be noted that many additional exemplary diagnostic or therapeutic ligands are described hereinbelow.

The invention also contemplates, particularly in therapeutic applications of the present invention, that the HDGF targeted agent may be administered in combination (e.g., before, during or after) with a second anti-hyperproliferative therapy to the subject. Such combination therapy will generally be a chemotherapy, a radiotherapy, a gene therapy, a molecular targeted therapy or a surgery, or any combination of the foregoing. An exemplary but non-limiting list of such chemotherapy includes the application of gemcitabine, taxol, cisplatin or carboplatin. In other embodiments, the second anti-hyperproliferative therapy is a molecular targeted therapy, such as an EGF, VEGF, IGF, PDGF, DNA methyltransferases, HDAC targeted therapy (e.g., as exemplified by therapeutic products such as Avastin®, Erbitux®/Cetuximab, Tarceva®, Herceptin®, IGFBP, ZD6474, ZD2171, Su11248, BAY43-9006, decitabine, SAHA, and the like). In the surgical context, the HDGF therapy could be considered either a pre- or post-adjuvant therapy where, for example, the HDGF therapy is employed as a pre-adjuvant to reduce tumor size or swelling prior to surgery, or as a post-adjuvant, where the HDGF therapy is employed to, in effect, “sterilize” the tumor bed or surrounding tissues post surgically.

It is contemplated that the present invention will find application in the context of hyperproliferative cells and disorders generally, examples of which include rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis. However, it is contemplated that the invention will find its greatest utility in the treatment of cancer, particularly those cancers whose cells overexpress HDGF. Non-limiting examples of the foregoing include melanomas, liver cancers, colorectal cancers, pancreatic cancers, NSCLCs, SCLC, esophageal cancers, stomach cancers, SCCHN. However, the inventors contemplate that the invention will be applicable to the treatment of virtually any other cancer, without regard to whether the cancer overexpresses HDGF, including by not limited to retinoblastoma, astrocytoma, glioblastoma, gum, tongue, leukemia, neuroblastoma, breast, renal, bone, testicular, ovarian, mesothelioma, cervical, gastrointestinal, lymphoma, brain, colon, sarcoma and bladder cancers.

It is envisioned that the HDGF therapeutics or diagnostics of the present invention may be administered by any convenient or appropriate route, depending on the procedure. Particularly preferred are systemic, local, topical or regional routes (although oral routes are not excluded). The agents may, for example, be administered parenterally, intravenously, intraperitoneally, topically, via inhalation, intra-abdominally or intra-tumorally. The formulation of the agents will thus typically depend on the intended route of administration.

In still further embodiments, the invention is directed to a method of assessing a cell suspected of being a cancer cell to determine the oncogenic potential of the cell comprising assessing the HDGF expression of the cell through the use of an HDGF antibody or a nucleic acid capable of determining HDGF expression (e.g., through an ability to bind HDGF mRNA). The method may be a method of assessing or prognosticating the clinical outcome of a cancer patient. In preferred embodiments, the method will include obtaining a tissue sample from a cancer patient or a suspected cancer patient, contacting cells of the sample with the HDGF antibody to assess HDGF expression in said cells. The method may alternatively include contacting nucleic acids from such cell using an HDGF nucleic acid probe, such as in the context of a DNA chip or microarray.

In antibody detection methods, HDGF expression may most conveniently be assessed by means of IHC. However, other techniques well known to those of skill, such as but not limited to ELISA, Western blot, MALDI, and the like, are useful alternatives. In the context of IHC analysis, the inventors have determined that a useful approach is to determine a “labeling index.” A labeling index is defined as the weighted mean of percentage of tumor cells displaying nuclear immunoreactivity (calculated by counting the number of HDGF positive tumor cells among at least 1000 tumor cells for each tissue section) multiplied by the degree of the staining intensity (1, 2, or 3, defined as weak staining, moderate staining, or strong staining, respectively). Typically, a labeling index of greater than or equal to about 185 exhibited in cells of a patient indicates a lower probability of 5 year survival as compared to patients whose cells exhibit a mean labeling index of less than about 185, although patients whose tumor had an HDGF labeling index of between about 158 and about 184 also showed lower survival than those whose tumors had and HDGF labeling index of less than about 158.

Furthermore, in clinical studies carried out by the inventors, it was found that serum could be employed for HDGF diagnostic purposes. In these studies, the sera from healthy individuals, the HDGF levels ranged from non-detectable up to about 750 pg HDGF per 100 μl serum whereas the levels in sera from patients with early stage NSCLC ranged from about the upper end of the normal levels up to about 2 ng per 100 μl serum, but could be as high as 4 ng per 100 μl serum. Thus, it is contemplated that for cancer diagnosis, prognosis, screening or treatment evaluation of cancer, particularly (although not limited to) NSCLC, a serum level of less than about 750 pg/100 μl serum will correlate with “normal” HDGF levels, whereas higher levels, particularly those in the nanogram/100 μl serum, will be indicative of a cancer; it should be appreciated, thus, that the level change may serve as an indicator of disease burden and/or the success or failure of the therapeutic regimen employed. Measurements can be carried out by any known immunologic techniques for measuring antigen levels. However, ELISA assays are preferred, and a convenient ELISA assay for carrying out serum measurements of HDGF is described hereinbelow.

Particularly preferred embodiments of the invention employ the diagnostic/prognostic applications of the invention in the context of an NSCLC or SCLC cell. Particularly with respect to NSCLC, the inventors have determined, for example, a strong correlation between HDGF expression and survival.

In still further embodiments, the invention is thus directed to monoclonal antibody compositions (and compositions of the underlying hybridomas) wherein the monoclonal antibody binds to and neutralizes the biological action of native HDGF. The monoclonal antibody may be an IgG, IgM, IgA, IgD or IgE. Exemplary IgG monoclonal antibodies described herein include those designated C1, H3, L5-9, C4, I4, D5 or A2, with antibodies C1 or H3 being particularly preferred. Antibody derivatives such as Fab′, Fab, F(ab)s, DAB, Fv or scFv are also contemplated. Further, it is contemplated that the most preferred antibodies will be chimeric, humanized or human anti-HDGF antibodies, such as chimeric, humanized, or human versions of one of the foregoing listed antibodies. It is also contemplated that monoclonal antibody compositions in accordance with the invention will include antibodies that selectively bind both a HDGF homolog and HDGF per se.

In further embodiments, the antibodies of the composition may be linked to an effector molecule or a reporter molecule. In the case of a reporter molecule, particularly preferred is an enzyme, a radiolabel, a hapten, a fluorescent label, a phosphorescent molecule, a chemiluminescent molecule, a chromophore, a luminescent molecule, a photoaffinity molecule, a ligand, a colored particle or biotin.

In other embodiments, the antibodies are linked to an effector molecule, including but not limited to a toxin, an apoptotic molecule, an antitumor agent, a therapeutic enzyme or a cytokine.

In other embodiments, the invention is directed to compositions comprising an siNA having the ability to target and down regulate the expression of HDGF or an HDGF family member or homologue. Typically, such siRNAs will comprise from 18 to 30 nucleobases, and more preferably 20 to 23 nucleobases. Exemplary siNAs are siRNAs that incorporate SEQ ID NO:47 or SEQ ID NO:48, or an siRNA-encoding DNA that encodes SEQ ID NO:47 or SEQ ID NO:48. A particularly preferred example of the former is referred to herein as HDGF-siRNA-1 or HDGF-siRNA-2. In the case of DNAs that are designed to express the siRNA, the DNA molecule may be comprised in a viral vector, such as an adenoviral, AAV, retroviral or lentiviral vector.

Preferably, particularly for clinical applications, the antibody or siNA is comprised in a pharmaceutically acceptable carrier, and may be appropriately aliquoted and titered and sterilized and placed into a sterile, sealed container, such as a vial or ampule, in an appropriate concentration. Particularly in the case of an siNA, the siNA may be formulated in a viral or non-viral (or both) formulations. Exemplary non-viral delivery platforms include lipid-based delivery platforms such as liposomal or emulsion based vehicles. In either case (i.e., whether an antibody or siNA), the pharmaceutical preparation is formulated depending on the application, for example, for parenteral, intravenous, topical, inhalation, etc.

As used herein, a “HDGF-targeting therapeutic” is defined herein as a compound which inhibits the function of (e.g., binds, reduces the activity of, or inactivates) and/or down-regulates (e.g., reduces the expression or biological function of) HDGF.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

A “subject” or “patient” is used interchangeably herein, which refers to a vertebrate, preferably a mammal, more preferably a human.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. FIG. 1A. Illustration of the degree of sequence conservation and homology of HDGF as among various species, as well as within the HDGF family. Over 90% conserved regions are found among mammals. FIG. 1B. The highest homology is seen in the first 98 amino acids among the family members.

FIGS. 2A-B: FIG. 2A, Effect of HDGF down-regulation on anchorage-independent cell growth of A549 and H226 cells, as measured by soft agar assay (×40 magnifications). The table below FIG. 2A represents the counts of colonies for each of the four cell lines and P values of statistical analysis. FIG. 2B, Invasion capability of A549 cells and H226 cells measured by an in vitro cell invasion system (×100 magnifications).

FIGS. 3A-B: FIG. 3A, Western blots showing expression of HDGF protein in eight NSCLC cell lines and two immortalized normal bronchial epithelial cell lines. β-Actin (ACTB) served as protein loading control. The lower panel shows relative expression level of HDGF quantified based its β-Actin level. FIG. 3B, Down-regulation of HDGF protein expression induced by HDGF-siRNA-1 in A549 cells (48 and 72 h after siRNA administration) and in H1944, H358, and H226 cells (72 h after siRNA administration). ACTB served as protein loading control. Lane 1, treated with Lipofectamine alone; lane 2, treated with 100 nM negative control siRNA; lane 3, treated with 100 nM HDGF-siRNA-1; lane 4, treated with 100 nM HDGF-siRNA-2.

FIGS. 4A-B: Effect of HDGF down-regulation on anchorage-dependent cell proliferation of A549 cells, as measured by MTT assay (FIG. 4A). A microelectronic cell sensor system was also used (FIG. 4B); the three lines represents cells treated with Lipofectamine alone, cells treated with 2 nM HDGF-siRNA-1, and cells treated with 100 nM HDGF-siRNA-1. Flowcytometry was also performed 72 h after siRNA administration on these groups of cells. Values are means ±standard deviations.

FIGS. 5A-B. FIG. 5A. Expression of HDGF, SERPINE2, GLO1, and AXL before and after treatment with lipofectamine alone (Lanes 1 and 4), 100 nM control-siRNA (Lanes 2 and 5), and 100 nM HDGF-siRNA-1 (Lane 3 and 6) in A549 and 11226 cells measured by northern blot analysis. FIG. 5B. shows the top 15 genes down-regulated after HDGF-siRNA-1 treatment measured by Affymetrix U133A chip.

FIG. 6. Effects of HDGF down-regulation on A549 NSCLC xenograft tumor growth. The tumor growth curves represent cells treated with Lipofectamine alone, Lipofectamine plus 100 nM negative control siRNA, and Lipofectamine plus 100 nM HDGF-siRNA-1, respectively as labeled in the panel. Results are expressed as mean tumor volume (calculated from five mice). The error bars show upper 95% confidence intervals.

FIGS. 7A-C. FIG. 7A. demonstrates graphically the effect of the indicated HDGF monoclonal antibodies C1, H3 and L5-9 on tumor growth in A549 xenograft nude mouse model. FIG. 7B. sets forth the therapeutic effect in terms of statistically significant (P<0.05) decreases in tumor weight, and FIG. 7C. illustrates the treated tumors themselves.

FIG. 8. Tumor growth in A549 xenograft model treated with single and combination of agents indicated in the right box. G, gemcitabine; A, Avastin®; H, H3 antibody.

### DESCRIPTION OF ILLUSTRATIVE EMBODIMENTS

### A. Introduction

The present invention arises out of the inventors' discovery that HDGF, or one of its homologues, can be used as a selective target for hyperproliferative cell therapies. In certain embodiments, a siNA or an antibody, antibody fragment or single-chain antibody that selectively affects HDGF (i.e., binds, reduces the function of and/or reduces the expression of HDGF) are be used to treat a hyperproliferative cell disorder such as cancer.

HDGF is a heparin-binding growth factor originally purified from media conditioned with the human hepatoma cell line HuH-7 and can stimulate proliferation of Swiss 3T3 cells (Nakamura et al., 1989). Its precise function is unclear, but HDGF is known to be highly expressed during the early development of many tissues, including cardiovascular (Everett, 2001), kidney (Oliver and Al-Awqati), and liver (Enomoto et al., 2002). Although lacking the secretory sequence present in most secretory proteins (von Heijne, 1986), HDGF has been shown to act as a potent exogenous mitogen for HuH-7 hepatoma cells (Nakamura et al., 1994), COS-7 kidney cells (Nakamura et al., 1994), aortic vascular smooth muscle cells (Everett et al., 2000), and endothelial cells (Oliver and Al-Awqati, 1998). As deduced from the cDNA clone of HDGF, the amino acid sequence contains 240 residues with a motif homologous to the consensus sequences of a bipartite nuclear localization sequence and a DNA-binding PWWP motif, suggesting that the protein translocates to the nucleus and binds to DNA.

However, agents that alternatively or additionally target other HDGF family members or homologues are within the scope of the invention. Included within the definition of HDGF, therefore, are HDGF family members such as HDGF from species other than humans (e.g., chimpanzee, canine, murine, rat, bovine, etc.), as well as other members of the HDGF family of proteins, including the HDGF-related proteins (“HRPs”). Exemplary HRPs include HRP-1, HRP-2, HRP-3, HRP-4 and p52/75 or LEDGF (lens epithelium-derived growth factor). Examples of the foregoing that have been listed on sequence databases include canineHRP-1, ratHRP-1, humanHRP-2, murineHRP-2, chimpHRP-2, canineHRP-3, murineHRP-3, ratHRP-3, chickenHRP-3, bovineHRP-4, humanP52/p75, murine P52/p75, rat P52/p75, bovine P52/p75 and chicken P52/p75. (Izumoto et al., 1997; Ikegame et al., 1999; Abouzied et al., 2004; Dietz et al., 2002; Singh et al., 2002).

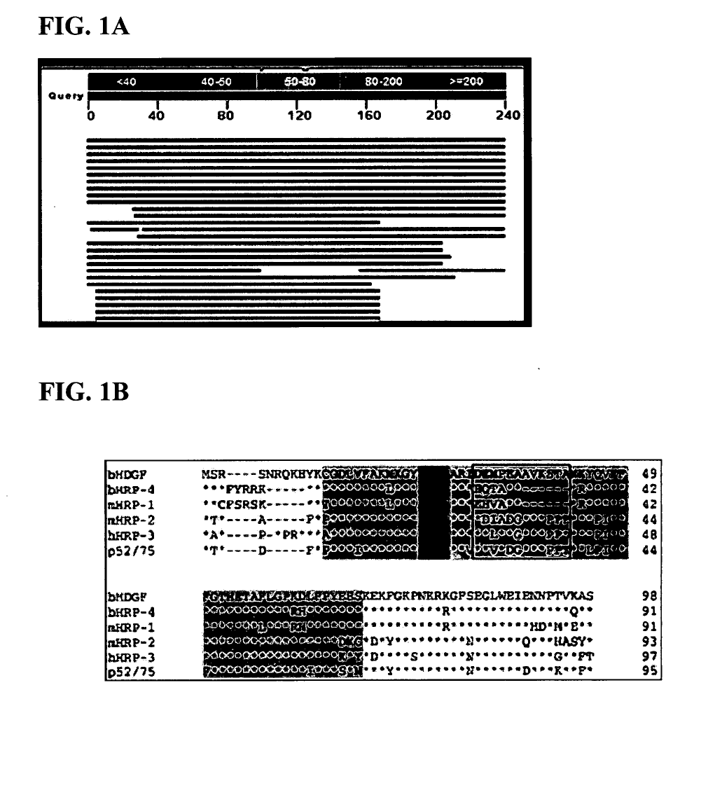

As can be seen in FIG. 1, the sequences of the HDGF family are highly, over 90%, conserved as between human, bovine, rat, mouse and chimpazee, with most of the differences occurring at the C-terminus. Furthermore, the bottom panel of FIG. 1 demonstrates that the highest homology of the family members is in the first 98 amino acids. The shaded parts coincide with defined heparin binding pocket, suggesting these members share heparin binding capability. The highly conserved region between amino acids 81-98 of the members suggests a potential receptor binding capacity of the proteins.

The sequences for the foregoing exemplary HDGF and HRP species can be found on the NCBI database as follows:
1. Human HDGF mRNA (NM_{—}004494) as reported in Nakamura et al., 1994, is set forth in SEQ. ID NO:1, the sequence of the human protein (NP_{—}004485) is set forth in SEQ ID NO:2.
2. HDGF from other species:
   a) P. troglodytes: XM_{—}513894, XP_{—}513894 (chimpanzee) (SEQ ID NO:3);
   b) C. familiaris: XM_{—}849818 XP_{—}854911 (canine) (SEQ ID NO:4);
   c) M. musculus: NM_{—}008231 (mouse) (SEQ ID NO:5);
   d) R. novegicus: NM_{—}053707 (rat) (SEQ ID NO:6);
e) B. Taurus: AJ237996 (bovine) (SEQ ID NO:7);
3. HRP-1 or “hepatoma derived growth factor-like 1”—no human homologs yet known:
   a) murine mRNA (NM_{—}008232) (SEQ ID NO:8);
   b) murine protein (NP_{—}032258.1) (SEQ ID NO:9);
4. HRP-1 from other species:
   a) C. familiaris: XM_{—}848364, XP_{—}853457 (canine) (SEQ ID NO:10)
   b) R. norvegicus: NM_{—}133549 (rat) (SEQ ID NO:11)
5. HRP-2 or “HDGF Related Protein-2” (note that murine sequence does not match the sequence reported in Dietz et al., 2002, though the Accession number does match);
   a) Human mRNA (NM_{—}001001520) (SEQ ID NO:12)
   b) Human Protein (NP_{—}001001520) (SEQ ID NO:13)
   c) M. musculus: NM_{—}008233 (murine) (SEQ ID NO:14)
   d) P. troglodytes: XM_{—}512288, XP_{—}512288 (chimpanzee) (SEQ ID NO:15)
   e) C. Familiaris: XM_{—}542154, XP_{—}542154 (canine) (SEQ ID NO:16)
   f) R. norvegicus: NM_{—}133548 (rat) (SEQ ID NO:17)
6. HRP-3 or HDGFRP-3
   a) human mRNA (NM_{—}016073) (SEQ ID NO:18)
   b) human protein (NP_{—}057157) (SEQ ID NO:19)
   c) C. familiaris: XM_{—}536208, XP_{—}536208 (canine) (SEQ ID NO:20)
   d) M. musculus: NM_{—}013886 (mouse) (SEQ ID NO:21)
   e) R. norvegicus: NM_{—}145785 (rat) (SEQ ID NO:22)
   f) G. gallus: XM_{—}413841, XP 413841 (chicken) (SEQ ID NO:23)
7. HRP-4 as reported in Dietz et al., 2002: B. Taurus AJ237666 (bovine) (SEQ ID NO:24).
8. P52/p75
   a) human mRNA (AF098483) (SEQ ID NO:25)
   b) human protein (AAC97946) (SEQ ID NO:26)
   c) M. musculus (AAH43079) (murine) (SEQ ID NO:27)
   d) R. norvegicus (AA032951) (rat) (SEQ ID NO:28)
   e) B. taurus (AAM90841) (bovine) (SEQ ID NO:29)
   f) G. gallus (Q5XXA9) (chicken) (SEQ ID NO:30)

HDOF expression, typically at high levels, has been observed in several cancers. HDGF expression has also been identified, for example in colorectal tumorigenesis (Lepourcelet et al., 2005), melanoma (Bernard et al., 2003) and liver cancer (Yoshida et al., 2003; Hu et al., 2003). Furthermore, the inventors have investigated the role of HDGF in non-small cell lung cancer (NSCLC) and squamous cell carcinoma of the head and neck (SCCHN) and found that the protein is frequently overexpressed in these tumors. Indeed, in an analysis of 121 primary SCCHN lesions it was found that 83 (69%) expressed high levels of HDGF. The inventors have also identified HDGF overexpression in small cell lung carcinoma (SCLC), pancreatic cancers, and it is contemplated that HDGF overexpression plays a role in liver, colorectal, stomach, melanoma and esophageal cancers. However, useful applications of the present invention are not limited to targeting tissues or tumors that overexpress HGDF or HDGF family members. This is due to the fact that underlying defects in the HDGF pathway are most likely responsible for upregulation of HDGF expression in the foregoing tumors, and since such defects may not always be manifested in HDGF overexpression.

Accordingly, it is contemplated that the present invention will find application in the context of hyperproliferative cells and disorders generally, examples of which include rheumatoid arthritis, inflammatory bowel disease, keloid formation, osteoarthritis, leiomyomas, adenomas, lipomas, hemangiomas, fibromas, vascular occlusion, restenosis, atherosclerosis, pre-neoplastic lesions (such as mouth, prostate, breast, lung, adenomatous hyperplasia, prostatic intraepithelial neoplasia, and the like), carcinoma in situ, oral hairy leukoplakia, or psoriasis. However, it is contemplated that the invention will find its greatest utility in the treatment of cancer, particularly those cancers whose cells overexpress HDGF. Non-limiting examples of the foregoing include melanomas, liver cancers, colorectal cancers, pancreatic cancers, NSCLCs, SCLC, hepatocarcinoma, esophageal cancers, stomach cancers, SCCHN. However, the inventors contemplate that the invention will be applicable to the treatment of virtually any other cancer, without regard to whether the cancer overexpresses HDGF, including by not limited to retinoblastoma, astrocytoma, glioblastoma, gum, tongue, skin, eye, prostate, leukemia, neuroblastoma, breast, renal, bone, testicular, ovarian, mesothelioma, cervical, gastrointestinal, lymphoma, brain, colon, sarcoma and bladder cancers.

A list of nonexhaustive examples of this includes extension of the patient's life by any period of time; decrease or delay in the neoplastic development of the disease; decrease in hyperproliferation; reduction in tumor growth; delay of metastases; reduction in the proliferation rate of a cancer cell, tumor cell, or any other hyperproliferative cell; induction of apoptosis in any treated cell or in any cell affected by a treated cell; and a decrease in pain to the patient that can be attributed to the patient's condition.

Nevertheless, it is contemplated that HDGF targeting will find its most beneficial application in the context of tumors and hyperproliferative tissues that overexpress, i.e., are “high” expressers of, HDGF. One convenient means of identifying tissues and cancers that are high or over-expressers of HDGF is through the use immunhistochemistry (IHC) techniques. In such IHC applications, the tissue or cancer cell is subjected to immunological binding using antibodies specific for HDGF (or a selected HDGF family member), and the degree of binding assessed by means of a label, such as by staining intensity employing an enzymatic tag. A particularly preferred method employed by the inventors is through determining a labeling index, or LI (intensity level multiplied by the percentage of positively staining cells). Typically, a LI of greater than about 185 is considered to be a high or over-expressor, whereas a LI of less than 185 is considered to be a low expresser.

In most somatic or quiescent non-tumor cells, such as normal lung or vascular smooth muscle cells, HDGF expression is often so low as to be virtually undetectable, or only barely detectable, by IHC techniques, and will often appear to stain just above background. Such tissues are believed to be useful negative controls, and, for example, may be assigned an LI number of 1 to 50. Most non-cancerous tissues, such as bronchial epithelial cells, will express a low level of HDGF, wherein the LI number will no more than about 100, and typically lower. In cancerous tissues, the LI is generally more than about 100. Among 98 primary NSCLC analyzed, only 3 (3%) had an LI of about 100, and all others had a much higher LI, with a mean LI of about 185. Other non-tumor tissues may be employed as controls as well. However, it will be appreciated that use of a control tissue or cell line is not an absolute requirement in that any degree of detectability of HDGF expression, particularly if detectable by IHC techniques, will be indicative of some degree of HDGF overexpression and will identify those tissues and cancers that will be of particular relevance in the practice of the present invention.

Of course, assessment of HDGF expression, for the purposes of the present invention, is not limited to IHC techniques and can include any other technique known to those of skill for determining protein expression levels. One such example is mRNA analysis, e.g., using gene chips or microarrays, designed to compare HDGF mRNA expression is a suspected tissue as compared to a control such as normal lung or vascular smooth muscle cells. Other useful examples would include, but are not limited to, quantitative RT-PCR for HDGF, an HDGF family member or an HDGF pathway gene (see, e.g., Table 1). Thus, the levels of proteins or expression of HDGF regulated down-stream gene may also be measured and serve as indicators of HDGF levels in tissues.

The present invention also contemplates targeting the HDGF receptor in a manner that prevents association of the HDGF molecule, and thus prevents HDGF proliferative activity. While a cell surface HDGF receptor has not been definitively identified, it has been shown that HDGF truncation mutants missing amino acids 81-100 (missing the so-called “HATH81-100 region), have cell surface binding activity (i.e., they bind to the unknown HDGF cell surface receptor) yet have no intrinsic proliferative or mitogenic activity (Abouzied et al., 2005). Such truncation mutants, or other “antagonist” molecules that bind the HDGF cell surface receptor but exhibit no agonist properties, are considered to be candidates for applications in the context of the present invention.

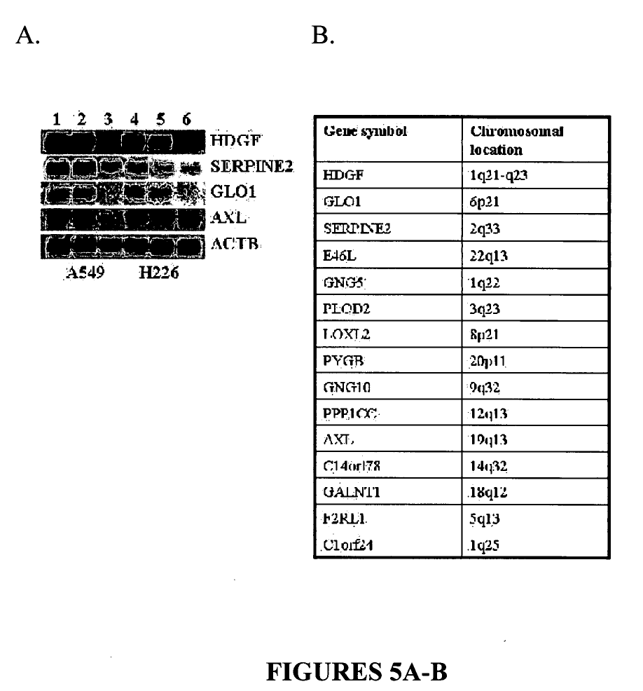

Furthermore, it is contemplated that other genes and gene products in the HDGF proliferation pathway may be targeted in accordance with the present invention. Such genes include in particular GLO1, SERPINE2, AXL or any of the other genes that have shown by the inventors to down regulate upon down regulation of HDGF (see Table 1 and FIG. 5).

### B. Therapies and Diagnostics Involving HDGF

The inventors contemplate that the down-regulation or inhibition of HDGF action will find particular application in the therapy of hyperproliferative disorders, particularly cancerous, precancerous or non-cancerous neoplasms. Most preferably, such down regulation or inhibition of HDGF action is accomplished through the application of therapeutically effective amount of an antibody (including antibody fragments, single chain antibodies, humanized and recombinant versions of the foregoing, and the like) or of a small inhibitory nucleic acid (“siNA”) specifically designed to reduce the expression of HDGF, and HDGF family member or one or more of the HDGF pathway genes (see Table 1 and FIG. 5).

In view of the studies set forth herein, it will be further appreciated that the HDGF antibodies and nucleic acids will also find substantial applications in diagnostic or prognostic embodiments. This will include, but not be limited to, assessing HDGF expression patterns, following the course of therapy or surgical procedures, in staging and profiling of tumors, predicting clinical outcomes and survival probabilities, in proteomic-assisted marker identification, and the like.

As noted above, exemplary monoclonal antibodies described herein include those designated C1, H3, L5-9, C4, I4, D5 or A2, with antibodies C1 or H3 being particularly preferred. Nevertheless, the inventors contemplate that antibody derivatives such as Fab′, Fab, F(ab)s, DAB, Fv or scFv will find useful application in accordance with the invention. Further, it is contemplated that the most preferred antibodies will be chimeric, humanized or human anti-HDGF antibodies, such as chimeric, humanized or human versions of one of the foregoing listed antibodies. Accordingly, as used herein, the term “antibody” or “monoclonal antibody” is intended to include any of the foregoing, including but not limited to IgG, IgM, IgA, IgD and IgE antibodies, as well as antibody derivates including but not limited to Fab′, Fab, F(ab)s, DAB, Fv or scFv antibodies.

With respect to the most preferred antibodies, C1 and H3, a preferred nucleic acid sequence encoding the C1 heavy chain is set forth in SEQ ID NO:31, and a preferred amino acid sequence of the C1 heavy chain is set forth SEQ ID NO:32; a preferred nucleic acid sequence encoding the C1 light chain is set forth in SEQ ID NO:33 and a preferred amino acid sequence of the C1 light chain is set forth in SEQ ID NO:34; a preferred nucleic acid sequence encoding the H3 heavy chain is set forth in SEQ ID NO:35 and a preferred H3 heavy chain amino acid sequence is set forth in SEQ ID NO:36; and a preferred nucleic acid sequence encoding the H3 light chain is set forth in SEQ ID NO:37 and a preferred H3 light chain amino acid sequence is set forth in SEQ ID NO:38.

With respect to the foregoing sequence information, it should be noted that only the mature variable regions of the heavy chains and light chains are included, which is the information needed by those of skill to recombinantly prepare a C1 or H3 antibody.

With respect to the most preferred antibodies, C1 and H3, the variable heavy chain and the variable light chain sequences can have a signal peptide. A preferred nucleic acid sequence encoding the C1 heavy chain with a signal peptide is set forth in SEQ ID NO:39, and a preferred amino acid sequence of the C1 heavy chain with the signal peptide is set forth SEQ ID NO:40; a preferred nucleic acid sequence encoding the C1 light chain with a signal peptide is set forth in SEQ ID NO:41 and a preferred amino acid sequence of the C1 light chain with a signal peptide is set forth in SEQ ID NO:42; a preferred nucleic acid sequence encoding the H3 heavy chain with a signal peptide is set forth in SEQ ID NO:43 and a preferred amino acid sequence of the H3 heavy chain with a signal peptide is set forth in SEQ ID NO:44; and a preferred nucleic acid sequence encoding the H3 light chain with a signal peptide is set forth in SEQ ID NO:45 and a preferred amino acid sequence of the H3 light chain with a signal peptide is set forth in SEQ ID NO:46.

1. HDGF Antibodies

Certain aspects of the invention relate to one or more antibodies which selectively bind HDGF. These antibodies may be used to treat a cancer (e.g., a melanoma, a liver cancer, a colorectal cancer, a pancreatic cancer, a lung cancer, NSCLC, a head or neck cancer). Further these antibodies may be used to evaluate expression of HDGF in a tissue, such as a cancerous or precancerous tissue.

As used herein, the term “antibody” is intended to refer broadly to any immunologic binding agent such as IgG, IgM, IgA, IgD and IgE. In certain embodiments, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow and Lane, 1988).

In certain embodiments, the HDGF antibody is a monoclonal antibody. Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

“Humanized” antibodies are specifically contemplated in the present invention, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's disease are likewise known and such custom-tailored antibodies are also contemplated

A “chimeric” antibody is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

a. Methods for Antibody Production

HDGF selective antibodies may be prepared using techniques well known in the art. For example, the methods for generating monoclonal antibodies (MAbs) generally begin along the same lines as those for preparing polyclonal antibodies. Briefly, a polyclonal antibody is prepared by immunizing an animal with a LEE or CEE composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

In order to generate a more vigorous immune response and aid in the production of antisera, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or down-regulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, CD40 or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal may then be fused with cells of an immortal mycloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). cites). For example, where the immunized animal is a mouse, one may use P3-X63/Ag8, X63-Ag8.653, NS1/1.Ag 4 l, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine mycloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine mycloma SP2/0 non-producer cell line.

Methods for generating hybrids of antibody-producing spleen or lymph node cells and mycloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused mycloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The mycloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

Monoclonal fully human antibodies may be produced using transgenic animals, such as XenoMouse which includes germline-configured, megabase-sized YACs carrying portions of the human IgH and Igkappa loci, including the majority of the variable region repertoire, the genes for Cmicro, Cdelta and either Cgamma1, Cgamma2, or Cgamma4, as well as the cis elements required for their function (Green, 1999). The IgH and Igkappa transgenes were bred onto a genetic background deficient in production of murine immunoglobulin. The large and complex human variable region repertoire encoded on the Ig transgenes in XenoMouse strains support the development of large peripheral B cell compartments and the generation of a diverse primary immune repertoire similar to that from adult humans. Immunization of XenoMouse mice with human antigens routinely results in a robust secondary immune response, which can ultimately be captured as a large panel of antigen-specific fully human IgGkappa mAbs of sub-nanomolar affinities. Monoclonal antibodies from XenoMouse animals have been shown to have therapeutic potential both in vitro and in vivo, and appear to have the pharmacokinetics of normal human antibodies based on human clinical trials.

b. Antibody Conjugates

The present invention further provides antibodies that selectively bind HDGF, generally of the monoclonal type, that are linked to at least one agent to form an antibody conjugate. In order to increase the efficacy of antibody molecules as diagnostic or therapeutic agents, the antibody may be covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins (e.g., gelonin, ricin, diphtheria toxin, etc.), apopototic molecules (e.g., granzymes such as granzyme B, IFNs, TNFs, KLAKK peptides, etc.), anti-tumor agents, therapeutic enzymes, radio-labeled nucleotides, antiviral agents, chelating agents, cytokines, growth factors, and oligo- or poly-nucleotides. Such antibody conjugates are often called immunotoxins, the preparation of which are generally well known in the art (see U.S. Pat. No. 5,686,072, U.S. Pat. No. 5,578,706, U.S. Pat. No. 4,792,447, U.S. Pat. No. 5,045,451, U.S. Pat. No. 4,664,911, and U.S. Pat. No. 5,767,072).

In contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

An HDGF antibody may be employed as the basis for an antibody conjugate. Sites for binding to biological active molecules in the antibody molecule, in addition to antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins.”

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236, 4,938,948, and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (II), with gadolinium being particularly preferred. Ions useful in other contexts, such as X-ray imaging, include but are not limited to lanthanum (II), gold (III), lead (II), and especially bismuth (III).

Radioactive isotopes for therapeutic and/or diagnostic application include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I may be preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nueleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'shannessy et al., 1987). This approach has been reported to produce diagnostically and therapeutically promising antibodies which are currently in clinical evaluation.

c. Immunodetection Methods

In still further embodiments, the present invention concerns immunodetection methods for binding, purifying, removing, quantifying and/or otherwise generally detecting HDOF. For example, it may be desirable in some instances to evaluate HDGF expression in a cancer prior to the administration of a HDGF-targeting therapeutic.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

In general, the immunobinding methods include obtaining a sample suspected of containing HDGF and contacting the sample with a first anti-HDGF antibody under conditions effective to allow the formation of immunocomplexes.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the HDGF produced antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any HDGF antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241, each incorporated herein by reference. Of course, one may find additional advantages through the use of a secondary binding ligand such as a second antibody and/or a biotin/avidin ligand binding arrangement, as is known in the art.

The HDGF selective antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes may be contacted with a third binding ligand or antibody that has binding affinity for the second antibody, again under effective conditions and for a period of time sufficient to allow the formation of immune complexes (tertiary immune complexes). The third ligand or antibody is typically linked to a detectable label, allowing detection of the tertiary immune complexes thus formed. This system may provide for signal amplification if this is desired.

One method of immunodetection designed by Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various diseases wherein a HDGF or HDGF homolog is expressed, for example, in a cancerous or pre-cancerous tissue. Here, a biological and/or clinical sample suspected of containing a specific disease associated HDGF expression product is used. However, these embodiments also have applications to non-clinical samples, such as in the titering of antigen or antibody samples, for example in the selection of hybridomas.

In the clinical diagnosis and/or monitoring of patients with various forms of disease, such as, for example, cancer, the detection of an over-expressed HDGF in comparison to the levels in a corresponding biological sample from a normal subject may be indicative of a patient with cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention may be employed in any immunodetection or therapy regimen known to one of ordinary skill in the art.

As detailed above, immunoassays, in their most simple and/or direct sense, are antibody binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

i. ELISA

In one exemplary ELISA, the HDGF selective antibodies of the invention are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA”. Detection may also be achieved by the addition of a second anti-HDGF, followed by the addition of a third antibody that has binding affinity for the second antibody, with the third antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

ii. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the at (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifigation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

2. HDGF Small Inhibitory Nucleic Acids (siNA)

The present invention provides small interfering nucleic acids (e.g., siRNA) that down-regulate the expression of HDGF. These HDGF siNA's may be administered to a subject in a pharmaceutical composition (e.g., a liposome or lipid-based composition) to treat a cancer.

“siNA,” as used herein, is defined as a small interfering nucleic acid. Examples of siNA include but are not limited to RNAi, double-stranded RNA, and siRNA. A siNA can inhibit the transcription of a gene in a cell. A siNA may be from 16 to 1000 or more nucleotides long, and in certain embodiments from 18 to 100 nucleotides long. In certain embodiments, the siNA may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. The siNA may comprise a nucleic acid and/or a nucleic acid analog. Furthermore, the siNA can be delivered directly to target cells, or may be delivered in the form of a DNA that will encode the siRNA once inside the cell. Such DNA delivery can itself be in the form of direct delivery of the DNA (e.g., formulated in a non-viral delivery platform) or may be delivered as a viral vector (e.g., adenovirus, AAV, retrovirus, lentivirus, and the like.) Typically, a siNA will inhibit the translation of a single gene within a cell; however, in certain embodiments, a siNA will inhibit the translation of more than one gene within a cell.

Within a siNA, a nucleic acid do not have to be of the same type (e.g., a siNA may comprise a nucleotide and a nucleic acid analog). siNA form a double-stranded structure; the double-stranded structure may result from two separate nucleic acids that are partially or completely complementary. In certain embodiments the present invention, the siNA may comprise only a single nucleic acid or nucleic acid analog and form a double-stranded structure by complementing with itself (e.g., forming a hairpin loop). The double-stranded structure of the siNA may comprise 16 to 500 or more contiguous nucleobases. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siNA (e.g., siRNA) are well known in the art. For example, siRNA and double-stranded RNA have been described in U.S. Pat. Nos. 6,506,559 and 6,573,099, as well as in U.S. Applications 2003/0051263, 2003/0055020, 2004/0265839, 2002/0168707, 2003/0159161, 2004/0064842, all of which are herein incorporated by reference in their entirety.

The two most preferred siRNAs for practice of the present invention includes what is referred to hereinbelow as siRNA-1 (SEQ ID NO:47) and siRNA-2 (SEQ ID NO:48).

Exemplary vehicles for delivery of nucleic acids (and proteins where desired) are lipid based vehicles. The lipid based vehicles for deliver of nucleic acids, and particularly siNAs, of the present invention may include virtually any lipid known to those of ordinary skill in the art for non-viral or viral based delivery of nucleic acids. For example, the lipid may a cationic lipid, such as DOTAP or DOTMA. It may be a commercially available vehicle such as Cremphor. In other embodiments of the present invention, the lipid is a neutral lipid, such as DOPE. The lipid may be included in a liposome. For example, the liposome may be a unilamellar or multilamellar liposome. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. In certain embodiments, a lipid component of a composition is uncharged or primarily uncharged. However, the carrier may also be a solvent or dispersion medium comprising but not limited to, water, ethanol, polyol (e.g., glycerol, propylene glycol, liquid polyethylene glycol, etc), alone or together with lipids (e.g., triglycerides, vegetable oils, liposomes) and combinations thereof.

### C. Pharmaceutical Compositions

Pharmaceutical compositions of the present invention comprise an effective amount of one or more HDGF-targeting agent or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one HDGF-targeting agent or additional active ingredient will be known to those of skill in the art in light of the present disclosure, as exemplified by Remington's Pharmaceutical Sciences, 19th Ed. Mack Printing Company, 1995, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The HDGF-targeting agent may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 19th Ed. Mack Printing Company, 1995, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a monoclonal antibody may be administered to a subject (e.g., a human patient) at a dose of about 1-25 mg/kg every 1 to 3 weeks. In certain embodiments, a siNA (e.g., a siRNA) may be administered at a dose of about 1-10 mg/kg at an interval of about daily to about weekly.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The HDGF-targeting agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the HDGF-targeting agent is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various amount of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fingi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Pharmaceutical compositions may also be in the form of lipid compositions. The lipid based vehicles for deliver of nucleic acids, and particularly siNAs, of the present invention may include virtually any lipid known to those of ordinary skill in the art for non-viral or viral based delivery of nucleic acids. For example, the lipid may a cationic lipid, such as DOTAP or DOTMA. It may be a commercially available vehicle such as Cremphor. In other embodiments of the present invention, the lipid is a neutral lipid, such as DOPE. The lipid may be included in a liposome. For example, the liposome may be a unilamellar or multilamellar liposome. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. In certain embodiments, a lipid component of a composition is uncharged or primarily uncharged.

In one embodiment, a lipid component of a composition comprises one or more neutral lipids. For example, the neutral lipid may be DOPE. In other embodiments of the present invention, the lipid is a cationic lipid. Examples of cationic lipids are discussed elsewhere in this specification. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges. In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

### D. Combination Therapies

In order to increase the effectiveness of a HDGF-targeting agent, it may be desirable to combine these compositions and methods of the invention with an agent effective in the treatment of hyperproliferative disease, such as, for example, an anti-cancer agent. An “anti-cancer” agent is capable of negatively affecting cancer in a subject, for example, by killing one or more cancer cells, inducing apoptosis and/or necrosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, altering a tumor stroma micro-environment, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with an HDGF-targeting agent effective to kill or inhibit proliferation of a cancer cell. This process may involve contacting the cell(s) with an agent(s) and the HDGF-targeting agent at the same time or within a period of time wherein separate administration of the HDGF-targeting agent and an agent to a cell, tissue or organism produces a desired therapeutic benefit. This may be achieved by contacting the cell, tissue or organism with a single composition or pharmacological formulation that includes both a HDGF-targeting agent and one or more agents, or by contacting the cell with two or more distinct compositions or formulations, wherein one composition includes a HDGF-targeting agent and the other includes one or more agents.

The terms “contacted” and “exposed,” when applied to a cell, tissue or organism, are used herein to describe the process by which a therapeutic construct of the HDGF-targeting agent and/or another agent, such as for example a chemotherapeutic or radiotherapeutic agent, are delivered to a target cell, tissue or organism or are placed in direct juxtaposition with the target cell, tissue or organism. To achieve cell killing or stasis, the HDGF-targeting agent and/or additional agent(s) are delivered to one or more cells in a combined amount effective to kill the cell(s) or prevent them from dividing.

The HDGF-targeting agent may precede, be co-current with and/or follow the other agent(s) by intervals ranging from minutes to weeks. In embodiments where the HDGF-targeting agent, alone or together with other agent(s), are applied in combination or separately to a cell, tissue or organism, one would generally ensure that a significant period of time did not expire between the time of each delivery, such that the HDGF-targeting agent and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the HDGF-targeting agent. In other aspects, one or more agents may be administered within of from substantially simultaneously, to about 1 minute to 5, 10, 20 or 30 minutes, to several hours (e.g., 2, 3, 5, or 10 hours), up to several days (e.g., 2, 3, 4, 5, 10 days), or event weeks or months apart, and any range derivable therein, prior to and/or after administering the HDGF-targeting agent or a previous dose of the HDGF-targeting agent.

Various combination regimens of the HDGF-targeting agent and one or more agents may be employed. Non-limiting examples of such combinations are shown below, wherein a composition comprising a HDGF-targeting agent is “A” and an agent is “B”

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the HDGF-targeting agent to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, gemcitabine, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, alimita, velcade, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference,” Goodman & Gilman's “The Pharmacological Basis of Therapeutics,” “Remington's Pharmaceutical Sciences,” and “The Merck Index, Eleventh Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

2. Radiotherapeutic Agents

Radiotherapeutic agents include radiation and waves that induce DNA damage for example, γ-irradiation, X-rays, proton beam therapies (U.S. Pat. Nos. 5,760,395 and 4,870,287), UV-irradiation, microwaves, electronic emissions, radioisotopes, and the like. Therapy may be achieved by irradiating the localized tumor site with the above described forms of radiations. It is most likely that all of these agents effect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes, for example, preventative, diagnostic or staging, curative and palliative surgery. Surgery, and in particular a curative surgery, may be used in conjunction with other therapies, such as the present invention and one or more other agents.

Curative surgery includes resection in which all or part of cancerous tissue is physically removed, excised and/or destroyed. It is further contemplated that surgery may remove, excise or destroy superficial cancers, precancers, or incidental amounts of normal tissue. Treatment by surgery includes for example, tumor resection, laser surgery, cryosurgery, electrosurgery, and miscopically controlled surgery (Mohs' surgery). Tumor resection refers to physical removal of at least part of a tumor. Upon excision of part of all of cancerous cells, tissue, or tumor, a cavity may be formed in the body.

Further treatment of the tumor or area of surgery may be accomplished by perfusion, direct injection or local application of the area with an additional anti-cancer agent. Such treatment may be repeated, for example, about every 1, about every 2, about every 3, about every 4, about every 5, about every 6, or about every 7 days, or about every 1, about every 2, about every 3, about every 4, or about every 5 weeks or about every 1, about every 2, about every 3, about every 4, about every 5, about every 6, about every 7, about every 8, about every 9, about every 10, about every 11, or about every 12 months. These treatments may be of varying dosages as well.

4. Genetic Therapy Agents

Gene therapy agents are another class of agents that are contemplated to be within the scope of the HDGF combination therapies of the present invention. One such example is the so-called suicide gene therapy employing the herpes simplex-thymidine kinase (HS-tK) gene, used in combination with a drug such as ganciclovir (Culver, et al., 1992). There are numerous other gene therapeutics that can be employed in combination with the present invention. Exemplary embodiments include the delivery of tumor suppressor or apoptotic genes or other therapeutic gene.

Any type of therapeutic nucleic acid is contemplated for inclusion in this aspect of the invention screening methods of the present invention. For example, the nucleic acid may be a deoxyribonucleic acid (DNA). In some embodiments, the deoxyribonucleic acid includes a therapeutic gene, such as a tumor suppressor gene, a gene that induce apoptosis, a gene encoding an enzyme, a gene encoding an antibody, or a gene encoding a hormone. For example, the therapeutic gene may be Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, mda7, FUS1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fins, trk, ret, gsp, hst, abl, EIA, p300, VEGF, FGF, thrombospondin, BAI-1, GDAIF, or MCC.

Other examples of therapeutic genes include the tumor suppressor genes at 3p21.3, including FUS1, Gene 26 (CACNA2D2), PL6, Beta*(BLU), LUCA-1 (HYAL1), LUCA-2 (HYAL2), 123F2 (RASSF1), 101F6, Gene 21 (NPRL2), and SEM A3.

In some embodiments, the nucleic acid is a DNA that encodes or is antisense RNA, such as antisense ras, antisense myc, antisense raf antisense erb, antisense src, antisense fms, antisense jun, antisense trk, antisense ret, antisense gsp, antisense hst, antisense bcl, or antisense abl. One of ordinary skill in the art would be familiar with antisense DNA, and other antisense DNA that may be included in the methods of the present invention. As with the previously described methods, the nucleic acid may be RNA, such as messenger RNA, antisense RNA, or interfering RNA. In some embodiments, the RNA further includes a ribozyme. In other embodiments, the nucleic acid is a DNA-RNA hybrid.

Other examples of therapeutic genes include the tumor suppressor genes at 3p21.3, including FUS1, Gene 26 (CACNA2D2), PL6, Beta*(BLU), LUCA-1 (HYAL1), LUCA-2 (HYAL2), 123F2 (RASSF1), 101F6, Gene 21 (NPRL2), and SEM A3. These genes, which play a major role in the pathogenesis of human lung cancer and other cancers, are addressed in detail in U.S. Patent Application. Pub. No. 20040016006 and U.S. Patent Application Pub. No. 20020164715, each of which is herein specifically incorporated by reference in its entirety.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Useful therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

5. Molecular Targeting Therapy

Another class of agents that are believed to be of particular relevance for use in therapeutic combinations of the present invention are the molecular targeting agents. These would include combination with agents that target other growth factors or growth factor receptors, including but not limited to agents that target VEGF (e.g., Avastin®) or its receptor (e.g., ZD6474, Su11248, BAY43-9006), agents that target EGF or its receptor (e.g., Cetuximab, Tarceva®, Iressa), IGF or its receptor (IGFBP, Herceptin®), bFGF, FGFR, TGF-alpha, IGF-IR, PDGF, PDGFR, TRAIL and its receptors, PI3K, farnesyltransferase, HIF-1, DNA methyltransferase, histone deacetylase, COX-2, and the like. From studies set forth below, it will be appreciated that the inventors have determined that a particularly advantageous therapeutic combination include combinations of anti-HGDF with VEGF directed therapies (particularly Avastin®) and/or Gemcitabine.

6. Compounds from Natural Products

Additional class of compounds derived from natural products is typically may also be effective in treating patients with proliferative diseases when these compound(s) are combined with anti-HDGF agent(s). These compounds would include but not limited to green tea extracts such as EGCG, resveratrol, curcumin, I3C, IP-6, fish oils, and their derivatives.

### E. Examples

The following examples are included to illustrate studies involved in the development of the invention and to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Immunology-Based Proteomic-Assisted Protein Marker Identification

To identify proteins differentially expressed between normal lung tissues and lung cancer tissues, the inventors developed a strategy based on the differences in the immunogenicity between normal and cancer tissues. The underlying premise is to use the natural immune response to generate antibodies against a battery of antigens (tumor tissues or normal tissues) in the hope that the differences in the types of proteins or the quantity of the proteins between normal tissues and tumor tissues will illicit antisera recognizing distinct protein pools. Two-dimensional gel electrophoresis (2-DE) was used to display the protein pools.

To increase the representation of candidate antigens, tissues from 10 patients with primary NSCLC (6 adenocarcinomas and 4 squamous cell carcinomas) were selected. The choice of primary tumor tissues was based on the following considerations: (1) comparative normal tissues with same genetic background can be used; (2) the differences in tumor microenvironment may be detected. Using this method, it was found that sera from mice immunized with normal lung tissues and sera from mice immunized with primary lung tumors had different antibody affinity to a few dozen proteins expressed in lung cancer cell lines (pool) and displayed on 2-DE. MALDI (matrix assisted laser desorption/ionization) mass spectrometry was used to determine the identities of these proteins after protenase digestion and peptide mapping. One protein spot recognized preferentially by sera from mice immunized with tumors was HDGF.

### Example 2

### HDGF is Overexpressed in NSCLC

Western blot analysis was performed to determine the HDGF expression patterns in NSCLC. Tissues or cells were either homogenized or harvested in a lysis buffer containing 50 mM HEPES, 1% Triton X-100, 10 mM NaF, 30 mM Na3P04, 150 mM NaCl, and 1 mM EDTA and freshly added 10 mM glycerophosphate, 1 mM Na3VO4, 20 μg/ml pepstatin A, 10 μg/ml aprotinin, 20 μg/ml leupeptin, and 40 μM microcystin-LR. Twenty micrograms of total proteins from each sample was separated in SD S-PAGE gels using a Bio-Rad Protean II apparatus and separated proteins in the gels were transfered to nitrocellulose membrane (Schleicher & Schuell BioScience, Keene, N.H.). The membranes were then blocked with 5% non-fat milk for 2 hours at room temperature followed by incubation with a rabbit anti-HDGF antibody (generously provided by Dr. Allen Everett of Johns Hopkins University) in 1:10000 dilutions at 4 C overnight. The immune reactive band was detected using a goat-anti-rabbit IgG-HRP conjugate (1:10000) (Jackson ImmunoResearch Lab, West Grove, Pa.) as the secondary antibody and SuperSignal West Pico Substrate (Pierce Biotechnology, Rockford, Ill.) as the detection agent. A mouse anti-actin monoclonal antibody (Sigma, St. Louis, Mo.) was used to normalize protein loading. A single band of molecular weight about 40 kDa was detected in all 13 NSCLC cell lines using the anti-HDGF antibody. Five of the cell lines, H358, H460, A549, H1299, and H1944, expressed higher levels of HDGF compared to other cell lines and only one cell line, H522, expressed a low level of HDGF. This data suggested that the expression levels of HDGF in NSCLC are variable and that the antibody used was specific to the denatured HDGF protein. To determine whether overexpression of HDGF occurred in primary tumors, five primary NSCLC specimens and their corresponding normal lung tissues were analyzed. Overexpression of HDGF was observed in 3 of the 5 primary tumors compared to the corresponding normal lung tissues. The presence of a moderately increased HDGF level in two corresponding normal lung tissues adjacent to the tumors may be due to the presence of premalignant lesions or even tumor cells not detected by routine histopathology. Alternatively, HDGF expression levels may be increased early in the carcinogenic process following heavy exposure to tobacco carcinogens. Indeed, all four normal lung tissues obtained from non-smoking patients with metastatic lung tumors from other organs had undetectable or only a trace level of HDGF protein.

### Example 3

### HDGF Expression Patterns in NSCLC

To determine expression patterns of HDGF in primary NSCLC, immunohistochemistry (IHC) analysis were performed using the anti-HDOF antibody described above. Tissue sections (4 μm thick) from formalin-fixed and paraffin-embedded tissue blocks were mounted on positively charged glass slides. Slides were baked at 60° C. for 1 h and then deparaffinized through a series of xylene baths. Rehydration was performed with graded concentrations of alcohol. To retrieve the antigenicity, tissue sections were treated with microwaves in 10 mM citrate buffer (pH 6.0) for 10 min. The sections were then immersed in methanol containing 0.3% hydrogen peroxidase for 20 min to block the endogenous peroxidase activity and incubated in 2.5% blocking serum for 30 min to reduce nonspecific binding. Sections were incubated overnight at 4° C. with the anti-HDGF antibody at a dilution of 1:4000, followed by incubation for 30 min with biotinylated antirabbit IgG (Vector Laboratories, Burlingame, Calif.). The sections were then processed using standard avidin-biotin immunohistochemistry according to the manufacturer's recommendations (Vector Laboratories, Burlingame, Calif.). Diaminobenzidine was used as a chromogen, and commercial hematoxylin was used for counterstaining. HDGF staining was observed in all tumor sections but at various intensities. Strong nuclear staining with minimal cytoplasmic staining was observed in many lung adenocarcinomas. The staining intensity was in general weaker and more variable in squamous cell carcinoma. The mean labeling index (the intensity level times percentage of positive cells) was 185 in 98 tumors from patients with pathologic stage I NSCLC. The ranges of labeling indices in each quartile from high to low were ≧211, 182.5≦HDGF<211, 158≧HDGF<182.5, and <158, respectively.

Thirty-four percent of the adenocarcinomas exhibited an HDGF labeling index more than 211 (highest quartile) compared to 15% of the squamous cell carcinomas, however, this difference was not statistically significant (P=0.09). Weak cytoplasmic staining was observed in some HDGF-positive squamous carcinoma cells. In normal lung tissues, vascular smooth muscle cells showed weak staining and were used as an internal control (intensity level 1). In normal lung tissues, the HDGF expression level was low. The HDGF expression level was not associated with age, sex, or race. In 40 tumors, the data for the proliferation marker Ki67 was available. The association between HDGF expression and the expression of Ki-67 was not statistically significant (corresponding coefficiency=0.1, P=0.52), suggesting that the role of HDGF in NSCLC is not simply in regulating cell proliferation.

In addition to the tumors from patients with stage I NSCLC, tumors from patients with locally advanced stages of disease were analyzed. Several interesting features have been noticed in these analyses. First, HDGF expression was detected in cells at all stages of the cell cycle except the cells in metaphase, suggesting a potential role of HDGF in the cell cycle regulation probably at the cell cycle entering phase. Second, the expression level of HDGF is higher at the tumor-invading front compared to other parts of the tumors; higher in the surviving tumor cells from the necrotic regions than other areas; and higher in the tumor cells of metastatic tumors than in the primary tumor sites. Interesting, a number of metastatic NSCLC tumors in the brain showed strong cytoplasmic HDGF staining. These data support the notion that higher expression of HDGF contributes to a more aggressive biologic behavior of the tumors.

### Example 4

### HDGF Expression Correlates with Clinical Outcomes in Patients with NSCLC

A total of 98 NSCLC patients were analyzed to assess a correlation between the level of HDGF expression in the primary tumors and survivals. It was found that patients whose primary tumors exhibited a high level of HDGF (when the mean labeling index of 185 was used as a cutoff point) had a significantly poorer overall survival (P<0.0001). The probability of overall survival at 5 years after surgery was 0.82 (95% confidence interval [CI]=0.72-0.93) for patients whose tumors showed a low HDGF labeling index (<185) compared with 0.26 (95% CI=0.16-0.43) for patients whose tumors showed a high HDGF labeling index (≧185). The probability of overall survival at 10 years after surgery was also lower for patients whose tumors had a high HDGF labeling index, but the difference was smaller compared to that at 5 years, probably due to an increase in non-cancer-related deaths over time in this patient population (median age of 62.5 years and 96% smokers). The striking difference in disease-specific survival at 5 years remained at 10 years after surgery and beyond between the high HDGF group and the low HDGF group. The probability of 5-year disease-specific survival was 0.92 (95% CI=0.84-1.00) for patients with a low HDGF labeling index compared with only 0.42 (95% CI=0.28-0.62) for the group with a high HDGF labeling index.

The disease-specific survival probability was highly significantly different between the two groups (P<0.0001). Because the labeling indices may be prone to some subjective variation in interpretation of staining intensity, particularly in tumors with scores around the mean level, we divided the patients into four groups based on quartiles of the HDGF labeling indices and compared the survival probabilities of the groups. As the HDGF labeling index increases, both overall survival (P<0.0001) and disease-specific survival (P<0.0001) decreases. When patients whose tumors exhibited a labeling index <158 (the lowest quartile) were compared with those whose tumors exhibited a labeling index ≧211 (the highest quartile), the difference in the survival probabilities was striking. At 5 years, none of the patients in the lowest quartile had died, while 84% of the patients in the highest quartile had died of any cause (95% CI=0.07-0.40) and 76% had died of lung cancer (95% CI=0.11-0.54). Of note, 8 of 9 patients who developed brain metastatses had higher HDOF levels in the primary lung tumors.

To determine whether HDGF expression level is an independent factor in predicting survival probability for patients with pathologic stage I NSCLC, multivariate analysis using the Cox model was performed. It was found that HDGF expression level was the only independent predictor of disease-specific survival probabilities (P<0.0001) among the parameters tested, including age, sex, race, smoking status, and tumor histology. When overall survival was analyzed, HDGF expression level remained the most significant independent predictor (P<0.0001); not surprisingly, age was also an independent predictor for overall survival (P=0.0002). Martingale analysis was performed and a linear correlation between HDGF level and survival became evident.

### Example 5

### siRNA Knock Down of HDGF

The present example is illustrates the preparation of siRNA molecules that target and “knock-down” the expression of HDGF, and the application of such siRNAs to demonstrate the molecular and anti-tumor effects of such knock-down or inhibition on tumor cells that overexpress HDGF, including effects on cell proliferation, anchorage-independent growth, cell invasion capabilities, tumor morphology and the expression of other genes in the HDOF pathway.

a. Materials and Methods

Cell Culture. NSCLC cell lines H226, H1944, H292, H157, A549, H596, H460, and H358 were obtained from ATCC and cultured in Dulbecco's modified Eagle's medium (DMEM) supplemented with 10% heat-inactivated fetal bovine serum (FBS). The normal human bronchial epithelial cell lines HBE1 and HBE3 (kindly provided by Dr. John Minna of The University of Texas Southwestern Medical Center, Dallas, Tex.) were cultured in keratinocyte serum-free medium with 25 μg/ml bovine pituitary extract and 0.2 ng/ml recombinant epidermal growth factor (Invitrogen, Carlsbad, Calif.).

siRNA and Knockout of HDGF Expression. The inventors selected two sites in the HDGF mRNA sequence as siRNA targets based on principles described previously (11). The targeted HDGF sequences, based on which the siRNAs were chemically synthesized by Ambion (Austin, Tex.), were 5′-AACCGGCAGAAGGAGUACAAA-3′ (siRNA-1; SEQ ID NO:47) and 5-AAAUCAACAGCCAACAAAUAC-3′ (siRNA-2; SEQ ID NO:48). The negative control siRNAs were purchased from Ambion. In vitro transfections were performed using Lipofectamine 2000 (Invitrogen, Carlsbad, Calif.) following manufacturer's protocols.

Cell Proliferation Analysis. Cells were plated onto 96-well plates at a density of 1×10⁴ cells per well with medium containing 10% FBS and incubated for 15 h. Cell numbers were determined at 0, 24, 48, and 72 h after transfection using the MTT-based CellTiter 96 cell proliferation assay (Promega, Madison, Wis.). ACEA RT-CES (ACEA Biosciences, San Diego, Calif.), a microelectronic cell sensor system, was used to confirm the number of living cells. NSCLC cells (1×10⁴) were seeded into each sensor-containing well (19.6-mm² surface with 150 μl of medium) of the microtiter plates. The electronic sensors provided a continuous (every 6 h), quantitative measurement of the cell index (reflect to the surface area covered by the cells) in each well. After 15 h of culture, the cells were transfected as described above. Cell growth was measured every 6 h for 72 h, and cell indexes were recorded for each well at all time points.

Cell Cycle Analysis. A549 cells were harvested by trypsinization 72 h after transfection and fixed with 70% ethanol. After RNase treatment, the cell cycle distribution was determined using a BD FACS Caliber flow cytometer and Cell Quest software (Becton Dickinson, San Jose, Calif.).

Anchorage-Independent Growth Assay. Twenty-four hours after transfection with siRNA, ˜2,000 cells in 1 ml of 0.3% agarose with DMEM were plated in each well on the top of existing 0.6% bottom agarose in six-well tissue culture plates in triplicate for each treatment condition. The plates were covered with 1 ml of medium with 10% FBS and incubated at 37° C. in a 5% CO₂ incubator for 3 weeks. The covering medium was replaced every week. At the end of 3 weeks, cell colonies >0.1 mm in diameter were counted under a microscopic field at magnifications ×40. Means were based on numbers from triplicate wells for each treatment condition and were analyzed using two-sided Student's t test.

In vitro Cell Invasion Assay. The in vitro invasion assay was carried out in BD BioCoat Matrigel invasion chambers (Becton Dickinson). After rehydration of the chambers, 1.1×10⁴ cells in 100 μl of the growth medium with 10% FBS were added into each of the upper chambers. Cells in the chambers were transfected with Lipofectamine alone or 100 nM HDGF-siRNA-1 in serum-free condition. Four hours later, the medium was replaced with fresh growth medium containing 10% FBS in the upper chambers whereas the lower wells contained serum-free medium. After 20 h, the medium in each of the lower wells was replaced with 750 μl of serum-free medium containing 30 μg/ml laminin (Sigma-Aldrich, St. Louis, Mo.). After an additional 24 h of incubation, the noninvading cells on the upper side of the chamber membranes were removed. The invading cells to the opposite side of the chamber membranes were examined. The invading cells on each of triplicate membranes were counted. Means were based on the numbers from the triplicate wells for each treatment condition and were analyzed using two-sided Student's t test.

Western Blot Analysis. Total proteins were loaded into each well on 10% SDS-polyacrylamide gels, separated by electrophoresis, and transferred to Hybond-P PVDF membrane (Amersham Biosciences, Buckinghamshire, UK). The membranes were probed with a rabbit polyclonal anti-HDGF antibody (gift from Dr. Allen Everett of Johns Hopkins Hospital, Baltimore, Md.) followed by incubating with horseradish peroxidase-conjugated anti-rabbit Immunoglobulin G (Amersham Biosciences). Immunodetection was performed using the enhanced chemiluminescence Western blotting analysis system (Amersham Biosciences). β-Actin was used as protein loading control (monoclonal anti-β-actin antibody [Sigma-Aldrich].

Global Gene Expression Analysis. Total RNA was isolated from cells using the Qiagen RNeasy Mini kit (Qiagen, Valencia, Calif.). Ten μg total RNA was reverse-transcribed into double-stranded cDNA and then transcribed in the presence of biotin labeled-ribonucleotides, using the BioArray HighYield RNA transcript labeling kit (Enzo Laboratories, Farmingdale, N.Y.) as described by the manufacturer. The biotin-labeled cRNA was purified using RNeasy mini-column (RNeasy kit; Qiagen, Valencia, Calif.) and fragmented at 94° C. for 35 min in 1× fragmentation buffer (40 mMTris-acetate, pH 8.0, 100 mM KOAc, 30 mM MgOAc). Affymetrix U133A chips were used for gene expression analysis using the Affymetrix GeneChip system (Affymetrix, Santa Clara, Calif.).

The expression levels were extracted from positional dependent nearest-neighbor model developed by Zhang et al. (2003). Genes that are absent or always expressed at low levels were excluded from further analysis because variation in gene expression at low levels are usually not reproducible. The removed genes have mean Log Expression Level less than 7.1. Genes with standard deviation less than 0.06 were also removed. Comparisons were then performed between cells treated with lipofectamine alone and cells treated with 100 nM HDGF-siRNA-1.

Northern Blot Analysis. Total RNA (10 μg) was loaded in each lane. cDNA probes corresponding to HDGF, GLO1, SERPINE2, AXL, and actin were prepared using RT-PCR followed by cDNA purification and labeling.

In vivo Tumor Model. Athymic Swiss nu-nu/Ncr nude (nu/nu) mice, bred and maintained in our institutional specific pathogen-free mouse colony were used. Briefly, 4-week-old male nude mice were injected subcutaneously with 106 A549 cells in 100 μL of PBS at a single dorsal site. Three groups (5 each) of mice were tested. Group 1 were injected with A549 cells treated with Lipofectamine alone; group 2 were injected with A549 cells treated with Lipofectamine plus 100 nM HDGF-siRNA-1; group 3 were injected with A549 cells treated with Lipofectamine plus 100 nM negative control siRNA. Tumor size was measured every two days for 20 days. Tumor growth was quantified by measuring the tumors in three dimensions with calipers. The results were expressed as the mean tumor volume (n=5) with 95% confidence intervals. The statistical significance of differences in tumor growth was analyzed using Wilcoxon rank sum test.

Tumor Morphology and Ki67 Immunohistochemistry. Formalin-fixed and paraffin-embedded tissue sections were stained with hematoxylin and eosin (H&E) for morphologic examination. For Ki67 immunohistochemistry, an anti-Ki67 antibody (Lab Vision, Fremont, Calif.) was used. The expression signal was detected using standard avidin-biotin immunohistochemical techniques according to the manufacturer's recommendations (Vector Laboratories, Burlingame, Calif.).

b. Results and Discussion

Down-Regulation of Hepatoma-Derived Growth Factor Inhibits Anchorage Independent Growth and Invasion of Non-Small Cell Lung Cancer Cells. The inventors knocked down HDGF expression in NSCLC cells to determine the biologic consequences. Transfection with HDGF-specific small interfering RNA (siRNA) resulted in down-regulation of HDGF expression in four NSCLC cell lines. Down-regulation of HDGF resulted in no detectable effect on anchorage-dependent cell growth as determined with an MTT assay, a microelectronic cell sensor system, and flow cytometry. In contrast, cells transfected with HDGF-siRNA grew more slowly and formed significantly fewer colonies in soft agar than did cells treated with Lipofectamine alone or transfected with negative control siRNA. In an in vitro invasion assay, significantly fewer cells transfected with HDGF-siRNA than cells treated with Lipofectamine alone were able to invade across a Matrigel membrane barrier.

In an in vivo mouse model, A549 cells treated with HDGF-siRNA grew significantly slower than the cells treated with Lipofectamine alone or negative control siRNA. Morphologically, HDGF-siRNA treated tumors exhibited markedly reduced blood vessel formation and increased necrosis whereas the Ki67 labeling indices were similar to tumors treated with controls. These results indicate that HDGF is involved in anchorage-independent growth, cell invasion, and formation of neovasculature of NSCLC. These qualities may contribute to the HDGF-associated aggressive biologic behavior of NSCLC.

As noted, HDGF expression was consistently downregulated in a number of NSCLC cell lines, including A549, H1944, H350 and H226 cells; the HDGF protein level was substantially reduced 48 hours after transfection with 100 nmol/L HDGF-siRNA-1, whereas 100 nmol/L HDGF-siRNA-2 induced only a slight reduction of the protein in A549 cells; the effect lasted up to at least 72 hours after transfection. Using 100 nmol/L concentration of HDGF-siRNA-1, the HDGF protein level was similarly down-regulated in these cells. These results indicate that HDGF-siRNA-1 effectively and specifically down-regulated HDGF protein expression in NSCLC cells. Interestingly, the down-regulation did not change the proliferation rates nor the cell cycle distribution of the cells cultured in medium with 10% serum, suggesting the effect of HDGF in cell proliferation is minimal when other growth stimulating factors are present.

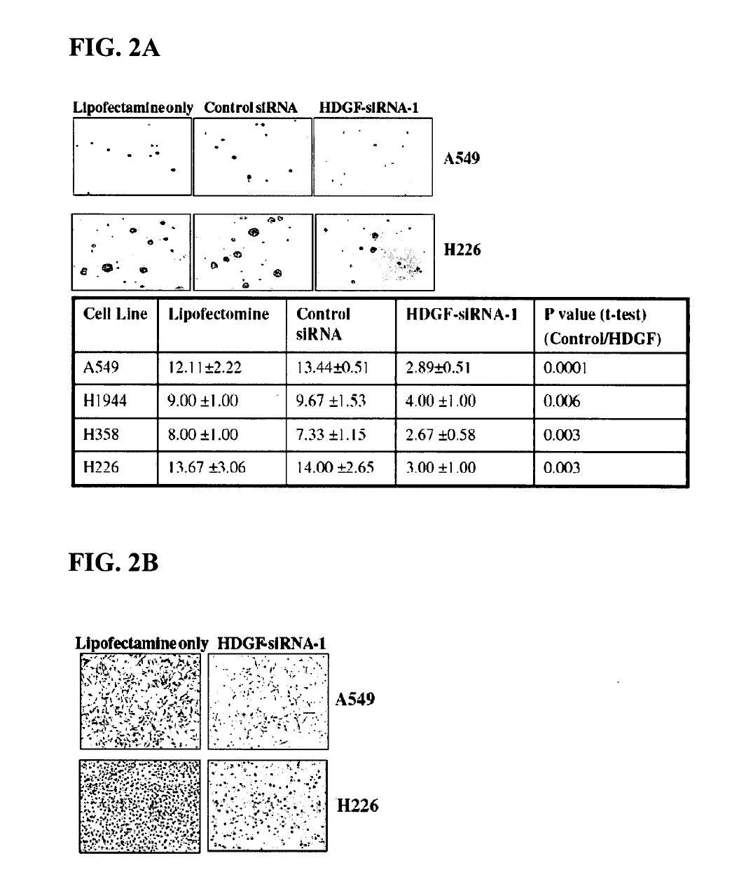

However, the down-regulation substantially reduced anchorage-independent growth of NSCLC cells. The effect of HDGF on anchorage-independent growth of the four NSCLC cell lines was analyzed using a soft agar growth assay. Three weeks after seeding, cells transfected with 100 nmol/L HDGF-siRNA-1 produced significantly fewer and smaller colonies than did cells treated with LipofectAMINE alone or transfected with negative control siRNA (FIG. 2A). The numbers (average of triplicate wells with three randomly selected fields per well) of colonies visible in a microscopic field at ×40 magnifications for the four cell lines are presented in an attached table in FIG. 2A. These results suggest that HDGF is involved in anchorage-independent cell growth, a feature of malignant transformation, of NSCLC cells. An invasion capability analysis was performed using Matrigel invasion chambers to determine the effect of HDGF on the invasion potential of the four cell lines. Transfection with 100 nmol/L HDGF-siRNA-1 resulted in significantly fewer cells invaded through the chambers in A549 and H226 cell lines. The number of cells transfected with HDGF-siRNA-1 that were invasive averaged 140 (140±73.65), whereas the number of cells treated with LipofectAMINE alone that were invasive averaged 759 (759±156.79; P=0.004) for A549; 126 (126±28) versus 516 (516±19; P=0.0001) for H226. Because H1944 and H358 did not invade in both controls and treated cells in these chambers, the effect of HDGF-siRNA-1 in these cell lines could not be determined. These results help to explain the increased rates of tumor relapse and distant metastasis in patients whose primary NSCLC tumors had a high level of HDGF after curative surgery.

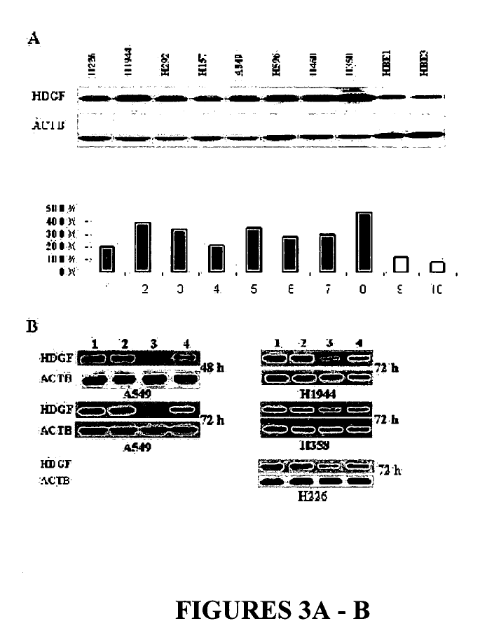

HDGF Is Highly Expressed in NSCLC. Western blot analysis using a polyclonal anti-HDGF antibody revealed that most of the NSCLC cell lines expressed high levels of HDGF, whereas the immortalized normal bronchial epithelial cell lines (HBE1 and HBE3) expressed low levels of HDGF (FIG. 3A). The inventors selected four cell lines (A549, H358, H226, and H1944) for further investigation. HDGF-siRNA-1 Knocks Out HDGF in NSCLC Cells. To determine the role of HDGF in NSCLC, the inventors used RNA interference (RNAi) strategy to down regulate the molecule. In A549 cells, the HDGF protein level was substantially reduced 48 h after transfection with 100 nM HDGF-siRNA-1, whereas 100 nM HDGF-siRNA-2 induced only a slight reduction of the protein; these effects lasted up to at least 72 h after transfection (FIG. 3B). Using 100 nM concentration of HDGF-siRNA-1, the protein level was similarly down-regulated in H1944, H358, and H226 cells. These results indicate that HDGF-siRNA-1 effectively and specifically down-regulated HDGF protein expression in a panel of NSCLC cells.

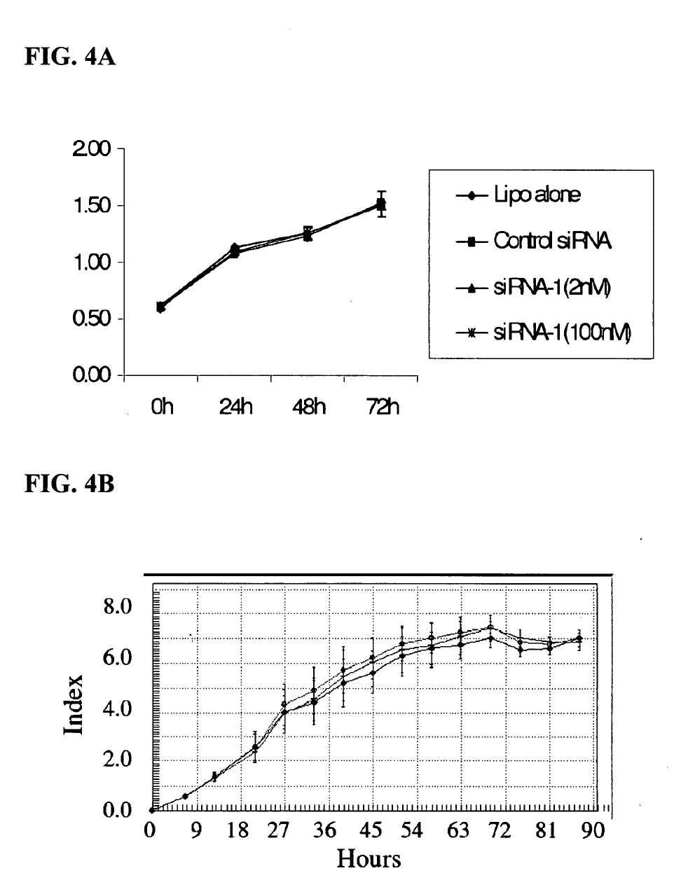

Down-Regulation of HDGF Has Minimal Effect on Anchorage-Dependent Growth of NSCLC Cells. The inventors next examined the growth curves of A549 cells transfected with 2 or 100 nM HDGF-siRNA-1 in the presence of 5% bovine serum. Results of the MTT assay showed that the growth curves of these cells were comparable to those of cells treated with Lipofectamine alone or transfected with negative control siRNA (FIG. 4A). These observations were confirmed by using a microelectronic cell sensor system (FIG. 4B). Similar results were obtained in H1944, H358, and H226 cells (data not shown). These data suggest that HDGF plays a minimal role in controlling anchorage-dependent growth in NSCLC cells in this culture condition.

To determine a role of HDGF in cell cycle regulation, the inventors performed flow cytometry analysis in A549 cells 72 h after transfection with 2 or 100 nM HDGF-siRNA-1. These results indicated that the cell cycle distributions of these cells were similar to those of cells treated with Lipofectamine alone or transfected with negative control siRNA.

Although HDGF can stimulate DNA synthesis and cell proliferation in vascular or bronchial epithelial cells has been previously reported (Everett et al., 2001; Kishima et al., 2002; Mori et al., 2004), the results are in consistent with our clinical observation that the expression levels of HDGF was not associated with Ki67 labeling indices in primary NSCLC (Ren et al., 2004). In fact, HDGF-mediated cell growth was observed only when the cells were cultured in serum-free condition (Mori et al., 2004; Everett et al., 2004); the presence of serum would have masked HDGF stimulation because of the effect of other growth stimulators in serum.

Down-Regulation of HDGF Reduces NSCLC Cells Capability to Invade. The inventors then used Matrigel invasion chambers to determine the effect of HDOF on the invasion potential of the four cell lines. Transfection with 100 nM HDGF-siRNA-1 resulted in significantly fewer invasive cells in A549 and H226 cell lines (FIG. 2B). The number of cells transfected with HDGF-siRNA-1 that were invasive averaged 140 (140±73.65) whereas the number of cells treated with Lipofectamine alone that were invasive averaged 759 (759±156.79) (P=0.004) for A549; 126 (126±28) vs. 516 (516±19) (P=0.0001) for H226. Because H1944 and H358 did not invade in both controls and treated cells in these chambers, the inventors were unable to determine the effect of HDGF-siRNA-1 in these cell lines.

Together with the soft agar experiments, these results may explain the increased rates of tumor relapse and distant metastasis in patients whose primary NSCLC tumors had a high level of HDGF after surgical removal of the tumors (Ren et al., 2004). An association between higher HDGF and poor clinical outcome has also been observed in patients with NSCLC by another group (Iwasaki et al., 2005) and in patients with primary hepatocellular carcinoma (flu et al., 2003) and melanoma (Bernard et al., 2003).

Genes down-regulated by HDGF-siRNA-1 treatment. Global gene expression profiles were analyzed using Affymetrix U133A and U133A plus chips to determine potential role of HDGF in gene expression. Gene expression profiles were analyzed in A549 and H226 cells treated with LipofectAMINE alone, 100 nmol/L scramble siRNA, and two doses (2 nmol/L and 100 nmol/L) HDGF-siRNA-1 at 48-hour time point. Combined data from repeated experiments, a small panel of genes whose expression was analyzed consistently correlated with the HDGF levels. It is interesting to note that down-regulation of expression was observed in a panel of genes after reduction of HDGF levels but no consistently up-regulated genes, suggesting HDGF is involved in enhancing gene expression in these cells.

Fifteen genes were found to be down-regulated ≧2-fold in HDGF-siRNA-1-treated A549 cells. Among the 15 genes, the expression of HDGF was down-regulated most dramatically (>4-fold) as expected. The overexpression of GLO1 and SERPINE2 has been implicated in cancer development and invasion. AXL, a receptor tyrosine kinase has been previously reported as being overexpressed in multiple types of cancers and linked to adverse clinical outcome in patients with cancer (Nakano et al., 2003). The down-regulation of GLO1, SERPINE2, and AXL in HDGF-siRNA-1 treated A549 and H226 cells was confirmed at mRNA level by using Northern blot analysis. Genes listed in Table 1 are consistently down-regulated after HDGF-siRNA treatment consolidated based on repeated experiments of the two NSCLC cell lines. A significant number of the genes have been implicated in cell invasion and regulation of extracellular compartment, suggesting an important role of HDGF in tumor stroma and ECM formation.

TABLE 1
Selected genes consistently regulated by HDGF

| | Chromosome | |
|---|---|---|
| Gene symbol | locus | Biologic function |
| HDGF | 1q21 | |
| GLO1 | 6p21 | Chemo-resistance |
| SERPINE2 | 2q33 | ECM, invasion |
| EGFR | 7p12 | Signaling, proliferation |
| GNG5 | 1p22 | Signaling, proliferation |
| PLOD2 | 3q23 | ECM, invasion |
| M-RIP | 7p11 | ECM, invasion |
| GMNN | 6p22 | Proliferation |
| HSPA5 | 9q33 | Chaperon |
| PPP1CC | 12q24 | Signaling |
| AXL | 19q13 | ECM, invasion |
| NNMT | 11q23 | Radiation-resistance |
| GALNT1 | 18q12 | O-glycosylation |
| NT5E | 6q14 | Differentiation |
| IER3 | 6p21 | Proliferation, apoptosis |

To ensure the relationship between HDGF expression and the gene expression observed in cell lines is not limited in cultured cells, the expression patterns of HDGF, AXL, GLO1, SERPINE2, and GNG5 were analyzed in 23 primary NSCLC tissues using real-time quantitative RT-PCR. Consistent with the HDGF knock down experiments, statistically significant correlations between the expression level of HDGF and the levels of AXL and GLO1 were observed. Tumors with higher HDGF levels had significantly higher levels of AXL and GLO1, suggesting the HDGF-mediated regulation of the genes also occurs in vivo. Similar trend was observed for SERPINE2 and GNG5 genes although the differences were not statistically significant, which may be due to the small sample size in the pilot study.

It is noted that GLO1 has been shown elevated in lung cancers (Sakamoto et al., 2001) whereas SERPINE2 has been suggested to play a role in invasion of pancreatic cancer cells (Buchholz et al., 2003). AXL, a receptor tyrosine kinase also in the list, has been reported overexpressed in multiple types of cancers (Wu et al., 2002; Meric et al., 2002; Wimmel et al., 2001) and linked to adverse clinical outcome in patients with cancer (Nakano et al., 2003). To confirm the down-regulation of GLO1, SERPINE2, and AXL in HDGF-siRNA-1 treated cells, the inventors performed northern blot analysis to compare the gene expression levels in A549 and H226 cells treated with HDGF-siRNA-1, siRNA control, and lipofectamine alone. The results are consistent with the microarray experiment (FIG. 5) and agree with the notion that HDGF is involved in regulation of expression of these genes.

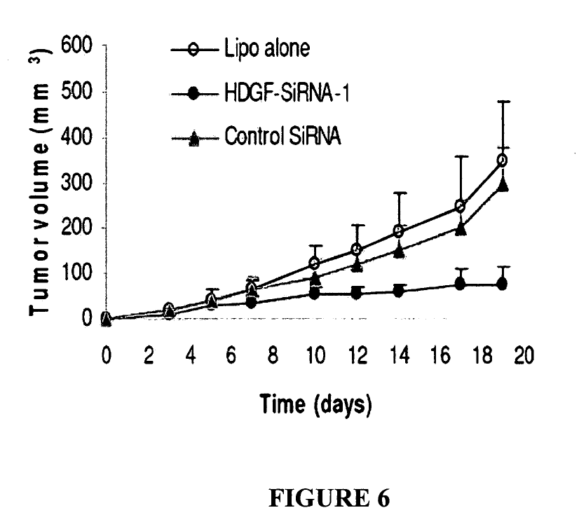

Down-Regulation of HDGF inhibits Tumorigenicity of NSCLC Cells in vivo. To further determine a role of HDGF in progression of NSCLC, the inventors performed an in vivo animal experiment. The inventors found that A549 cells transfected with 100 nM HDGF-siRNA-1 formed substantially smaller tumors in nude mice compared to those transfected with Lipofectamine alone or negative control siRNA (FIG. 6). The tumor volume for mice with cells transfected with the HDGF-siRNA was 76.27±39.06 mm³ compared to 345.64±135.67 mm³ or 295.33±80.53 mm³ for mice with cells treated with Lipofectamine alone or negative control siRNA, respectively (P=0.037 or P=0.018, respectively). Under light microscopy, the inventors observed a substantially reduced blood vessels in the HDGF-siRNA transfected tumors compared to the tumors derived from cells treated with Lipofectamine or negative control siRNA. Substantial tumor necrosis was observed only in tumors derived from cells treated with the HDGF-siRNA. Interestingly, Ki67 expression index, an indicator of cell proliferation, was similar in tumors of the three animal groups.

The in vivo animal experiment provides a strong support for the importance of HDGF in NSCLC and suggests that HDGF may be a target for treating NSCLC or preventing the development of lung cancer. The finding of reduced blood vessel formation in the HDGF-siRNA treated tumors suggests that HDGF plays a role in the neovasculature formation in vivo, which may be an important mechanism of HDGF in tumor development and progression of NSCLC. These results are consistent with previous reports supporting the role of HDGF in angiogenesis as a potent endothelial mitogen and regulator of endothelial cell migration by mechanisms distinct from those used by vascular endothelial growth factor (Everett et al., 2004; Okuda et al., 2003). The observed tumor necrosis is likely a consequence of the poor blood supply in these tumors. Consistent with our in intro and clinical observations, the lack of change in cell proliferation in the tumors indicates that HDGF plays a minimal role in the tumor cell proliferation for patients with NSCLC. Future studies will focus on the molecular mechanisms of HDGF-induced tumor development and progression as well as on strategies to down-regulate the protein or inhibit its function for potential therapeutic applications.

### Example 6

### Development of Monoclonal Antibodies with High Binding Affinity to Native HDGF

The polyclonal antibody used for IHC could not bind to native HDGF. Thus, to develop a useful therapeutic that could affect tumor control, it was deemed important to develop high-affinity monoclonal antibodies that had the ability to bind native HDGF. An exemplary approach that was employed by the inventors involved, first, generating full-length recombinant HDGF in bacteria. This bacterially expressed recombinant HDGF was then used the immunize mice to generate mature B cells secreting antibodies specifically recognizing HDGF. After fusing the B cells with myeloma cells and several rounds of screening/selection steps, single clones secreting high-affinity IgGs capable to bind native HDGF were identified. Clones designated H3, L5-9, C4, and C1 were selected for further testing because of their ability to immunoprecipitate HDGF, a feature of recognizing native protein, and their specificity to HDGF and/or its potentially modified forms. Antibodies from these clones were selected and tested using IHC. Similar staining patterns were obtained in a panel of tumor samples among these antibodies and between these antibodies and the previously mentioned polyclonal antibodies, indicating these antibodies recognize HDGF. All of the four monoclonal antibodies are IgG1 and have been adapted to serum-free conditions for scale-up production.

### Example 7

### HDGF Resides in Nuclear Compartment and Cytoplasm and is Secreted/Released to Extracellular Space by NSCLC Cells

In most studies, HDGF was found localized in nuclear compartment. However, HDGF was identified in conditioned medium suggesting it may be secreted or released from cells. One possibility of the lacking cytoplasmic staining is the common IHC conditions. Alternatively, the protein may be rapidly released from cells making the detection of protein in cytoplasm difficult. Taking advantage of the newly developed monoclonal antibodies, two sets of experiments were performed to determine the subcellular localization of HDGF and the secretion/releasing status of the protein. In one set of experiment, H3 was used for immunoprecipitation (IP) employing total cell lysates from H460 NSCLC cells. The antibody sufficiently pulled down over 90% of the cellular HDGF. Some potential HDGF binding proteins might have also co-precipitated with HDGF. We then extracted cytoplasmic proteins and nuclear proteins separately and performed IP as described above followed by 2-DE protein separation. Although the relative HDGF protein amount was higher in the nuclear than in the cytoplasm with equal amount of protein loading, HDGF was clearly presented in cytoplasm with distinct modification or homologues patterns between the two subcellular compartments. It should be noted that the total amount of cytoplasmic proteins is much higher than the nuclear proteins. Therefore, the absolute amount of HDGF in cytoplasm may actually be similar or even higher to that in nuclear.

Second set of experiment was designed to determine whether HDGF is secreted or released from NSCLC cells as well as the relative quantity of the secretion or release. Four NSCLC cell lines were tested and measured HDGF levels in supernatant of the cultures and the total cell lysates using pooled monoclonal antibodies in order to reveal all the modified or homologues of HDGF. Because a serum-free culture condition was adapted in the experiment, no contamination from bovine serum was expected (bovine HDGF would migrate differently if exist). Cells from each of the cell lines secreted or released HDGF although three of the four NSCLC cell lines secreted or released substantially large amount of HDGF. It is interesting to note, however, there were different patterns of secretion or releasing. For example, H358 cells released only 40 kD HDGF whereas H1294 and SK cells released both 40 kD and the slower migrating protein.

### Example 10

### HDGF Neutralizing Antibodies Possess Anti-Tumor Activity

Considering the role of HDGF in cell invasion and ECM, in vivo animal models were employed to test whether the HDGF neutralizing antibodies have anti-tumor activity. In the first experiment, mixed monoclonal antibodies (equal amount of H3, C1, C4, and L5-9) were employed in the A549 xenograft model. A549 was derived from a patient with poorly differentiated lung adenocarcinoma and is widely used in both in vitro and in vivo experiments. Each nude mouse was injected with 4×10⁶ A549 cells subcutaneously. At day 6, when the tumors formed about 50 mm³ in size, the animals were randomized into two groups (5 per group). One group of mice was injected with 150 μg mixed antibodies intra-peritoneal and the other group was injected with PBS. The treatment was repeated every other day for four times and the animals were sacrificed at day 20. It was observed that tumors in the antibody treated mice grew substantially slower than the tumors in PBS treated control mice. The average weight of tumors from the 5 mice treated with the antibody was 172±41 mg compared to 671±437 mg of tumors from PBS treated controls (P=0.037 by student T test). The treatment had no noticeable toxicity to the animals such as weight loss and moving activities. No gross abnormality was observed in major organs of the antibody treated animals.

To determine an anti-tumor effect of individual monoclonal antibodies, the CELLine™ system (BD Biosciences) was used to generate the amount of antibodies for each clone sufficient for our animal experiments. In our second experiment, we tested four individual monoclonal antibodies (H3, C1, C4, and L5-9) and used PBS and M31, an IgG1 antibody without known antigen, as controls. Cell inoculation was similar to the experiment described above, 7 days after subcutaneous injection of A549 cells, the animals with established tumors were randomized into one of the 6 groups. Besides PBS group, which was given 200 μl PBS every IP injection, all the other groups were given a 500 μg of individual antibody as boost dose followed by 250 μg/injection every three days for four times. Tumor sizes were measured every three days. All the animals were sacrificed at day 22 after tumor cell inoculation.

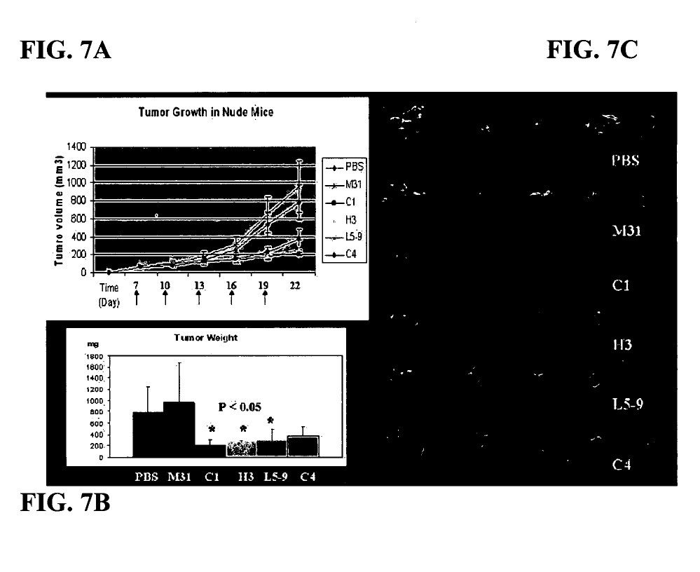

The results, shown in FIG. 7, demonstrated that, compared to PBS and M31 controls, while all of the four anti-HDGF antibodies showed some anti-tumor activity in terms of tumor growth inhibition, the most significant tumor growth inhibition was observed in mice treated with antibodies C1 and H3. The average tumor weights were 784±458 for PBS treated control group and 960±713 for M31 treated control group. In contrast, the average tumor weights were only 224±85 for C1 treated group and 266±22 for H3 treated group. These experiments demonstrate the anti-tumor activity of the antibodies and justify the selection of C1 and H3 for further investigation. Again, no noticeable treatment-related toxicity was observed in any of the treatment group. C1 and H3 antibodies were further tested in H460 xenograft model and had showed similar tumor growth inhibition with C1 antibody slightly better than H3.

To determine the distribution of the antibodies after intra-peritoneal injection in the animals, we injected fluoresce labeled M31 and H3 antibodies into nude mice and used a scanner to measure the antibody distribution 12 h after injection. Both M31 control IgG1 and H3 monoclonal antibody evenly distributed in the animals including the tumors, indicating the antibodies can reach to the tumors through intra-peritoneal injection and the lack of treatment toxicity is not due to the lack of drug distribution to the organs. To determine the antibody distribution in the tumor tissues and to compare the patterns of the distribution in the tumor tissues among the different antibodies, a specific anti-mouse IgG antibody was used to stain tumor tissue sections of tumors treated with different antibodies. As expected, no IgG was detected in tumors treated with PBS (nude mice do not produce antibodies) whereas mouse IgG was detected in tumors treated with M31, C1, or H3, indicating these antibodies were delivered to the tumor tissues. Interestingly, the M31 antibody molecules were mainly localized in extracellular compartment of the tissue whereas H3 entered into the cytoplasmic compartment of some tumor cells and C1 entered into both cytoplasm and nuclear mainly in the tumor cells.

To determine potential synergistic effects of combining the antibodies with other cancer therapeutic agents, the H3 antibody was tested in combination with gemcitabine, a commonly used chemotherapeutic agent, and Avastin®, a FDA approved VEGF neutralizing monoclonal antibody, in the A549 xenograft model. At day 7 after tumor cell inoculation subcutaneously, the animals were randomized into one of the 8 groups: M31 (6 mice), gemcitabine (5 mice), H3 (7 mice), Avastin® (5 mice), H3+gemcitabine (5 mice), 113+Avastin® (5 mice), Avastin®+gemcitabin (5 mice), and H3+ gemcitabine+Avastin® (7 mice). In this experiment, all the agents were given IP at day 7 and every 3 days thereafter for total 5 injections. H3 was given 250 μg per mouse/injection without boost at first injection; gemcitabine was given in a dose of 80 mg/kg/injection; and Avastin® was given 100 μg per mouse/injection.

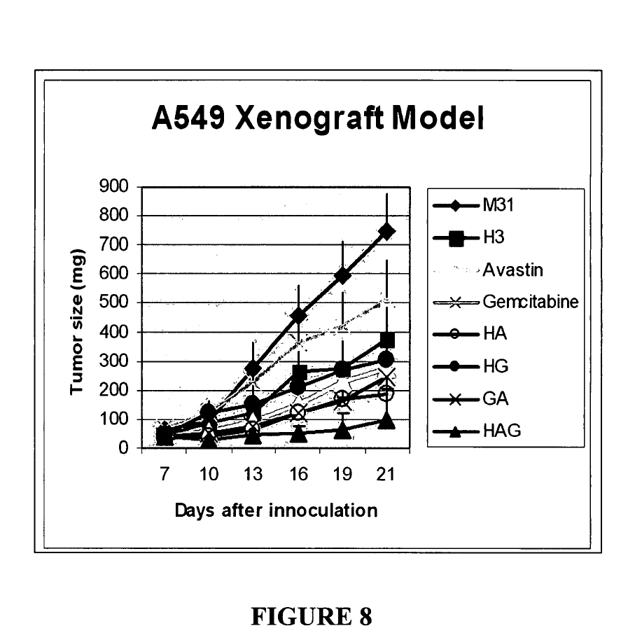

The results are illustrated in FIG. 8. While tumor growth inhibition was observed in all single agent groups, Avastin® showed the best inhibition followed by H3 whereas only modest tumor growth inhibition was observed in mice treated with gemcitabine. Importantly, the combinations appear to have better tumor growth inhibition than the single agents. The most significant inhibition was observed in the three agent combination. Compared to the average 750 mg tumor weight in M31 treated mice, the average tumor weight was only 100 mg in the three agents treated mice. Tumors in three of the seven animals treated with the combination shrunk to less than 10 mg at the time the animals were sacrificed. In the experiment, no animal weight loss was observed and there was no indication of toxicity in any of the treatment groups, indicating that the treatment including the combinations was not toxic to these animals.

An interesting observation that was made during these studies illustrate certain differences between monoclonal antibodies C1 and H3 in their tumor growth inhibition, although both C1 and H3 were effective in inhibiting tumor growth in A549 xenograft model and H460 (large cell lung cancer) xenograft model. In both cases, the C1 antibody showed slightly better tumor growth inhibition. The two antibodies were tested using what was believed at the time to be the MiaPaca-2 pancreatic cancer xenograft model. In this tumor model, a better tumor growth inhibition by administrating H3 antibody was observed. This observation led to the use of H3 in the initial combination experiment described above. To determine the expression of HDGF in the cell line, a Western blot analysis was performed using C1 and H3 antibodies. Compared with NSCLC cell lines, C1 and H3 did not detect HDGF on the Western blots but L5-9 detected the protein with faster migration. To determine HDGF expression levels in pancreatic cancers, 7 additional pancreatic cancer cell lines were analyzed. All the 7 cell lines expressed high-level HDGF recognizable by H3 antibody but not the putative “MiaPaca-2” line.

An immunoprecipitation assay was then performed using H3 antibody to determine whether the antibody recognizes the native HDGF in the cells, which is important to explain why the antibody had inhibited the tumor growth in the xenograft model. H3 strongly bound to the native HDGF of the tumor cells. Because the protein migrated faster than the HDGF from NSCLC cell lines, the HDGF cDNA from “MiaPaca-2” cells was sequenced. Surprisingly, the sequence matched perfectly to murine HDGF, indicating that the “MiaPaca-2” line was a murine and not a human tumor line. The origin of the cell line was traced and is now believed to be M109, a murine lung adenocarcinoma cell line from a mouse spontaneous lung cancer.

The foregoing discovery and analysis demonstrated that C1 and H3 antibodies recognize epitopes distinct from those recognized by L5-9 and C4 antibodies whereas the better growth inhibition in the mouse tumor by H3 antibody is probably due to its stronger binding affinity to the native mouse HDGF. Together, these data also indicate the possible difference in the protein binding sites between H3 and C1 and therefore a potential difference in their anti-tumor activity in different tumors and/or in combination with different therapeutic agents.

### Example 11

### Antiangiogenic Action of Anti-HDGF Antibodies

In order to directly study the antiangiogenic action of anti-HDGF antibodies, the xenograph tumors treated with the various agents and presented in the foregoing example (see FIG. 8) were studied following CD31 staining. CD31 antibody staining is generally regarded as a convenient means of visualizing microvessels in order to assess angiogenesis and anti-angiogenic action. Each of the tumors treated as described with respect to FIG. 8 were subjected to CD31 staining. The resultant stains demonstrated that although both Avastin® and H3 antibody substantially reduced numbers of microvessels in the tumors, H3 antibody appears to have a stronger role in reducing the size of the microvessls than Avastin®. In contrast, gemcitabine appeared to further increase the size of the microvessles in the tumors although the agent had a modest tumor inhibitory effect in the A549 model. Interestingly, both Avastin® and H3 antibody were found to reduce the number and size of the microvessels when combined with gemcitabine. Again, the H3 antibody showed a stronger effect in reducing the size of the tumor microvessels.

When the H3 antibody was combined with Avastin®, CD31 positive cells were virtually undetectable in the central part of the tumors. This is consistent with the enhanced tumor growth inhibition observed with this combination (FIG. 8). When gemcitabine was combined with both H3 antibody and Avastin®, the number and size of the microvessels were still further reduced in the tumors compared to combining with either one of the two antibodies. This regimen resulted more than 85% inhibition of the tumor growth including tumors in 3 of the 7 mice shrank from approximately 50-75 mg to 9-13 mg at the end of the treatment. These data indicate that the tumor inhibiting effect of H3 antibody is at least in part due to inhibition of tumor microvessel formation and this effect is distinct with VEGF neutralization antibody Avastin®. The use of H3 antibody with chemotherapeutic agent gemcitabine substantially inhibited gemcitabine induced increase of tumor microvasculature and enhanced antitumor activity, which can be further strengthened by combining with other anti-angiogenic agents such as Avastin®.

### Example 12

### Anti-HDGF Antibodies Induced “Hollow Effect” in Pancreatic Cancer Model

Anti-HDGF antibodies were tested for a therapeutic effect in a xenograft model using metastatic L3.6 pancreatic cancer cells. After 10 days of tumor cell inoculation, mice were randomly selected into either M31 control antibody treatment, H3 antibody treatment, or C1 antibody treatment. The sizes of the tumors were between 100-200 mg at the time of treatment except three which grown bigger than 500 mg and were assigned to each group. Interestingly, the two mice with bigger tumors assigned to either H3 or C1 treatment developed liquidized central part once week after treatment but not the bigger tumor treated with M31 or any other small tumors. This phenomenon is similar to human clinical observations where some established primary lung cancers develop liquidized central part of the tumors after treatment with agents having anti-angiogenic activity such as sorafenib (BAY 43-9006). This is a hallmark feature of anti-angiogenic agents as a class. This result further supports the role of the anti-HDGF antibodies in anti-tumor angiogenesis as one of the therapeutic mechanisms. Indeed, the mice in the present study that were treated with anti-HDGF antibody exhibited a 70% increase of survival time compared to mice treated with M31 control antibody. These data support the role of anti-HDGF antibody in treating patients with pancreatic cancer.

### Example 13

### NSCLC Heterotransplant Model

One of the common concerns using xenograft models is its relevance to human therapeutic studies because the cancer cell lines are highly selected in vitro and may be not representative to primary tumors. Heterotransplant models are considered more representative of primary tumors but they are harder to establish. A panel of NSCLC heterotransplant models had been previously established and used to test common chemotherapeutic agents such as paclitaxel (Perez-Soler et al., 2001). The overall tumor take rate was 46% (95% CI, 3656%) in the initial inoculation. The histological morphology of all successfully heterotransplanted tumors was compared with that of the resected original tumors. There were no significant morphological differences between the tumors resected from the patients and the initial successful implants. The median time from the day of implantation from human to mouse to the day the tumor reached 10 mm in diameter was 11 weeks (range, 4-24 weeks), and the median weight doubling time, which corresponds to a 20% increase in diameter, was 18 days (range, 11-40 days). These values are longer than those observed with commonly used NSCLC xenografts and closer to those of human NSCLC tumors. All successfully heterotransplanted tumors can be subsequently transplanted several times to provide tissues for multiple experimentations.

### Example 14

### Prospective Study to Determine the Role of HDGF as a Prognostic Marker for Patients with NSCLC

Based on the cumulative studies set forth above, it is evident that HDGF expression levels in tumor cells determine biologic features of lung cancer and therefore can be used as a predictive marker. Indeed, in a retrospective study, the expression level of HDGF in patients with early stage NSCLC were found to correlate with patients' overall survival, disease-specific survival, and disease-free survivals. The difference in patients' survival between patients whose tumors expressed low level of HDGF and those whose tumors expressed high levels of HDGF is highly significant and striking, indicating a role of HDGF as a biomarker in risk assessment. Furthermore, the experimental data described above support the conclusion that HDGF is involved in tumor cell invasion and metastasis. Because HDGF is a novel therapeutic target, knowledge gained from this aim will help to determine a potential use of HDGF as a biomarker in patients' selection and/or monitoring treatment responses.

In the prospective study, patients will be identified from a prospective existing clinical research database of patients who undergo surgical resection through the Department of Thoracic and Cardiovascular Surgery at M. D. Anderson Cancer Center (MDACC). This unique database was established in 1997 and includes all patients who undergo thoracic surgical resection in the Department. Detailed demographic, clinical, and pathologic data are recorded prospectively using standardized data collection forms. Follow-up information is collected at each clinic visit. Using this database, patients will be identified that underwent complete NSCLC resection for their pathologic stages I to IIIA tumors, who had at least 3 year follow-up after surgery. The study will conform to standard institutional guidelines, with no gender or minority-based exclusionary criteria. In the past, the ethnic distribution of our patient population has been approximately 90% white, 5% black, and 5% others including Hispanic. The study has enrolled 282 patients treated between 1997 and 2001 with verified disease stage, available tumor tissues, and follow-up data. The population represents approximately 70% of all the stage I or II NSCLC patients treated during the period in the surgery department. The ethnic distribution of the patient population is similar to the general patient population at MDACC. Approximately 45% of the patients are females, which is consistent with the national trend to have an increased proportion of female lung cancer patients.

A uniform set of clinical variables, including gender, age, tumor histology, pathologic stage, preoperative clinical variables (performance status, weight loss, smoking status, alcohol use), date of disease recurrence, date and site of second primary tumors, date of death, or date of follow-up for patients who are still alive, and cause of death for deceased patients will be abstracted from the clinical database described above. The proposed sample size is 450.

The primary endpoint will be overall survival. Secondary endpoints are disease-specific survival and disease-free survival. The roles of HDGF will be assessed in each subgroup of the patients such as patients with squamous carcinoma or adenocarcinoma; patients who have recurrent disease or metastasis; and specific types of metastasis such as brain metastasis. Vital status and disease recurrence status will be obtained from the clinical research database described above, and when necessary, the medical record. For serum analysis, positive correlation between serum HDGF level and HDGF expression level in the tumors will be assessed while an association between the serum levels and clinical endpoints will also be examined.

For tumors, tissue sections (4 μm thick) from formalin-fixed and paraffin-embedded tissue blocks will be mounted on positively charged glass slides. Slides will be baked at 60° C. for 1 h and then deparaffinized through a series of xylene baths. Rehydration will be performed with graded concentrations of alcohol. To retrieve the antigenicity, tissue sections will be treated with microwaves in 10 mM citrate buffer (pH 6.0) for 10 min. The sections will then be immersed in methanol containing 0.3% hydrogen peroxidase for 20 min to block the endogenous peroxidase activity and incubated in 2.5% blocking serum for 30 min to reduce nonspecific binding. Sections will be incubated overnight at 4° C. with purified anti-HDGF monoclonal antibody H3 at 100 ng/ml concentration, followed by incubation for 30 min with biotinylated antirabbit IgG (Vector Laboratories, Burlingame, Calif.). The sections will then be processed using standard avidin-biotin immunohistochemistry according to the manufacturer's recommendations (Vector Laboratories, Burlingame, Calif.). Diaminobenzidine will be used as a chromogen, and commercial hematoxylin will be used for counterstaining. The HDGF labeling index is defined as the percentage of tumor cells displaying nuclear immunoreactivity (calculated by counting the number of HDGF positive tumor cells among at least 1000 tumor cells for each tissue section) multiplied by the degree of the staining intensity (1, 2, or 3, defined as weak staining, moderate staining, or strong staining, respectively). The weak staining in the smooth muscle cells of blood vessels will be used as an internal control and the basis of weak staining. For serum samples, we will quantify serum HDGF using ELISA method. We have established a reliable assay using C1 as the capture antibody and biotin-labeled H3 as the detection antibody. When we coated 40 ng C1 to each well of a 96-well plate and used 1 ng H3 to detect captured HDGF, we can detect as low as 150 pg HDGF. The recombinant HDGF generated standard curve will be used to quantify serum HDGF levels. In pilot studies, HDGF levels in sera from healthy controls were between non-detectable and 750 pg per 100 μl serum whereas the levels in sera from patients with early stage NSCLC ranged from about undetectable (but generally no less than about 750 pg) to more than 2 ng, but could be as high as 4 ng per 100 μl serum.

Tumor tissues from a minimum of 300 stage I/II NSCLC patients with complete resection and no adjuvant therapy and 150 stage III patients with complete resection will be analyzed. HDGF will be quantified by standard procedures specified above and the distribution will be examined by the BLiP plot (a versatile Box, Line, and Point plot; Lee et al., 1997). HDGF will be analyzed first as a continuous variable and appropriate transformation will be made if necessary so that the transformed variable will be more Gaussian distributed. Subsequently, HDGF can be dichotomized at the median value and patients can be classified into the low- or high-HDGF groups. Similarly, quartiles can be used to discretize HDGF into 4 expression groups to further characterize its prognostic values. The association of HDGF with demographic and medical variables such as age, gender, race, smoking/alcohol usage and histology, etc. will be examined by t-test, Wilcoxon rank-sum test, chi-square test, multiple linear regression, logistic regression analysis, and Pearson's or Spearman's correlation coefficients whenever appropriate. Similar analyses will be used to determine associations between serum HDGF levels and clinical outcomes. The association between HDGF expression in the tumors and HDGF serum level will be analyzed to determine their correlation.

With 450 patients, the prognostic effect of dichotomized HDGF on overall survival (estimated 5-yr survival for the entire group=45%) can be identified with at least 80% power if the hazard ratio between the high-HDGF and low-HDGF is 1.67 or greater with a two-sided 5% type I error rate. Similarly, the study will have at least 80% power to identify the prognostic effect of HDGF for disease-free survival (estimated 5-yr disease-free rate=40%), disease-specific survival (estimate 5-yr disease-specific survival rate=60%), SPT-free survival (estimated 5-yr SPT-free rate=70%), and brain-metastasis-free survival (estimated 5-yr brain mets-free rate=80%) with a hazard ratio of 1.61, 1.87, 2.19, and 3.26, respectively. It is noted that preliminary data showed that HDGF was highly significant (hazard ratio>5) in predicting overall survival and disease-specific survival.

Standard survival analysis methods will be applied to analyze time-to-event endpoints. Kaplan-Meier plots and 95% confidence intervals will be generated to estimate the event-free survival. Complementary to the survival curves, even charts (Lee et al., 2000) will be generated to give an effective visual display of the relationship among multiple timed events at the individual level. Cox regression model will be applied to study the prognostic effect of HDGF after adjusting by other potential confounding covariates. Residual analysis using the Martingale residual and Shoenfeld residual will be applied to check the goodness-of-fit of the Cox model. Appropriate transformation on variables and extended Cox model can be applied to ensure that the models provide adequate fit of the data (Therneau et al., 2000).

Based on the studies carried out by the inventors to date, it is expected that a significant percentage of HNSCC will have high HDGF expression, particularly the locally advanced tumors whereas the expression levels will be variable among the NSCLC specimens. Measurement of serum HDGF levels is a novel aspect of this study. However, it is expected from the studies conducted to date that a high level of serum HDGF will be detected in certain patients because our preliminary experiment showed that the serum HDGF level can be very high in patients with even early stage NSCLC but not in any of the healthy controls. It is possible the higher serum level is correlated to the protein expression levels in the tumors. Because the release of HDGF from tumor cells can vary substantially, the correlation between the tumor expression level and the serum protein level is expected to be perfect.

It is anticipated that patients whose tumors express higher level of HDGF and patients with higher serum HDGF levels have shorter overall disease-specific, and disease-free survivals than those whose tumors or sera have lower levels of HDGF. Such correlation may also be observed in certain subgroup analyses such as patients with different tumor histology and different tumor stages because of the large sample size of the study. However, subgroup analyses based on serum HDGF levels may have less power because of the samples may only be available in 50% of the patients. Nevertheless, a primary objective of analyzing the serum protein levels is to confirm its correlation with the tumor HDGF levels.

In a preliminary study, it was found that 8 of the 9 patients who developed brain metastasis had high-level of HDGF expression in their primary tumors. Of note, the single patient with low HDGF level (who developed brain metastasis) survived more than 5 years after the diagnosis of the brain metastasis, which is an extremely unusual case for patients with NSCLC. Together with our findings that NSCLC brain metastasis expressed high-level of HDGF and HDGF's role in NSCLC cell invasion, it is anticipated that patients whose tumors express higher level of HDGF will have a higher probability to develop brain metastasis. Among the 282 stage I NSCLC patients entered in one of the inventors' studies (medium follow-up 40 months), there were 14 confirmed brain metastasis. Therefore, with 450 patients (one third with locally advanced tumors) and longer follow-up proposed, more than 30 brain metastasis may be encountered.

Although IHC has been widely used in clinical pathology to determine protein expression levels, it is well-recognized that the method can generate variable results due to a number of factors including quality of the antibodies, sample preparation, adequate control, and staining interpretation. The HDGF antibody that will be used in this study, antibody C1, is highly specific and recognizes mainly HDGF migrated at 40 kD location on Western blot. The HDGF staining in IHC is mainly nuclear and is easily recognized. Several cell blocks will be generated from cell lines with different levels of HDGF expression including immortalized normal bronchial epithelial cell lines to be used as references for each batch of IHC experiment to ensure the consistency of the staining and scoring.

### Example 15

### Prospective Studies to Determine the Therapeutic Role of HDGF Neutralizing Monoclonal Antibodies in NSCLC Xenograft Models and to Reveal Potential Mechanisms of the Anti-Tumor Effects

The inventors contemplate that HDGF antibodies inhibit HDGF activity in lung tumors through neutralizing HDGF in ECM, cell surface, and even intracellular compartments and therefore, interfere with processes of tumor progression. Because HDGF is a novel therapeutic target, an added or synergistic effect may be achieved by combining the antibodies with therapeutic agents with distinct mechanisms.

As noted, overexpression of HDGF has been shown in multiple human cancers and correlates with a poor clinical outcome in cancer patients including NSCLC. HDGF may regulate expression of genes important in tumor cell invasion and formation of tumor microenvironment. Inhibition of HDGF either through down-regulation of the gene expression or neutralizing the growth factor substantially reduced the growth of lung cancers in xenograft models. An added or synergistic anti-tumor effect was observed when HDGF inhibition was combined with other cancer therapeutic agents. These results strongly suggest that HDGF is an important factor in lung cancer progression and may be a novel therapeutic target. The availability of neutralizing monoclonal antibodies against this novel target makes it possible to test the proposed novel hypotheses.

Production of purified C1 and H3 monoclonal antibodies: To perform multiple animal experiments proposed above, large amount of purified antibodies must be produced. Using CELLine™ system and protein G purification, the inventors are able to produce approximately 35 mg/L culture medium H3, 70 mg/L culture medium C1 and M31 IgGs with more than 95% purity within one month time frame. Such production capability will be sufficient to support our need for animal experiments.

To determine effect of tumor growth inhibition by C1 and H3 in multiple xenograft tumor models: Three NSCLC cell lines (A549, H226, and H460) will be selected in experiments proposed in the aim because they represent the three major histology subtypes of NSCLC, i.e. adenocarcinoma, squamous cell carcinoma, and large cell carcinoma, and the ability of the cells to form tumors in both subcutaneous and orthotopic models. Subcutaneous models have been traditionally used to test cancer therapeutic agents and are relatively simple in terms of procedures and observation of tumor growth. However, the models are frequently criticized for its potential relevance to primary tumors because the location of the inoculation (occasionally, lung cancers may metastasize to skin and these metastasized tumors do response to chemotherapy similar to their primary tumors). Orthotopic models are considered more relevant models because it mimics the anatomic locations of the primary tumors, which provides a microenvironment similar to primary tumors. However, the experimental procedures to generate orthotopic tumors are more complicated and more variations may be observed. It should be noted that whether therapeutic results generated from orthotopic models are better correlated to human tumors remains an unresolved issue at this time.

First, the C1 and H3 antibodies will be tested as single agents in the A549 subcutaneous xenograft model to select optimal dose which results in the maximal tumor growth inhibition but with minimal toxicity to the animals. The following doses will be tested in the model: 100 μg, 250 μg, 600 μg, and 1,500 μg per animal i.p. injection every 3 days. Two times of the proposed dose will be given in first injection to boost the dose. The treatment will start at about 7 days after tumor cell inoculation when the tumors are established and reach to 50-100 mm³. Besides tumor growth, animal weight change and other habits will be monitored to detect signs of toxicity. Major organs will be examined grossly and histopathologically. In a preliminary study, the inventors found no indication of any animal toxicity at 250 μg dose level using either C1 or H3 antibody. Together with experience of other antibody agents reported, it is reasonable to assume that C1 and H3 will be well tolerated at these dose levels proposed in the experiment. Relevant doses of M31 antibody, an IgG1 with no known antigen in human and murine cells, will be used as the antibody control in all the experiments. Ten animals in each treatment group will be tested to determine tumor growth and animal survival of each animal group. Because institutional animal protocol requires that animals with tumors larger than 1.5 cm³ be sacrificed, this criterion will be employed as the animal survival endpoint if the animals survive from other health problems. ANOVA analysis will be used to determine statistical significance between each treatment group by using the SAS procedure mixed with SAS version 6.12 software. Kaplan-Meier method will be used to compare animal survivals among the treatment groups.

Once the optimal doses are determined for each of the two antibodies based on the subcutaneous model, the doses will be tested in other two subcutaneous xenograft models (H460 and H226) and orthotopic tumor models of the same three cell lines. To establish orthotopic thoracic tumors, the mice will be inoculated with tumor cells by intra-thoracic injection with a 27-gauge needle. These mice will then be randomly divided into treatment groups (10 mice/group): I) M31, II) C1, and III) H3. Orthotopic tumors are usually established on the lungs or on the inner thoracic membrane 10-14 days after tumor cell inoculation. Before treatment, five mice from each group will be subjected to magnetic resonance imaging (MRI) analysis to establish tumor load and volume baselines. The mice will be given respected treatment (therapeutic antibodies or the control antibody). The animals with baseline MRI tumor measurement will undergo repeat MRI every 7 days after treatment start to determine tumor growth and spread in thorax in each treatment group. For MRI, mice will be given anesthesia for intubation and ventilation. The MRI protocol includes T2-weighted echo-planar imaging (EPI; TE=40 ms, TR˜16 s, 3×3-cm field of view, 1.25-mm slice thickness, 128×128 image matrix, 8 shots) with and without fat suppression, T1-weighted spoiled gradient echo (SPGR; TE=1.4 ms, TR˜1 s, 128×128 image matrix, 60-degree flip angle), and T1-weighted spin-echo (SE; TE=6.1 ms, TR˜1 s, 256×256 image matrix) pulse sequences.

All acquisitions are synchronized with respiratory and cardiac cycles, with data collection initiated during full expiration and at a consistent but arbitrary point in the cardiac cycle. For EPI, one shot is acquired from each slice during each breath. The fat suppression pulse is manually adjusted for each mouse using a one-pulse spectroscopic sequence with an additional fat suppression module that is identical to that in the EPI sequence. During SPGR and SE acquisitions, one phase-encoding repetition is collected from each slice during each breath. Tumor measurements are performed using Image J software (National Institutes of Health, Bethesda, Md.). In each EPI image containing a tumor, the periphery of the mass is traced, and the area of the drawn region is calculated. The areas are then multiplied by the slice thickness plus the skip distance to obtain the volume of each slice containing the object of interest. The slice volumes are then summed. Assuming a tissue density of 1 g/cm³ (0.001 g/mm³), to derive weight, the volume of the object of interest in mm³ is multiplied by 0.001 g/mm³. The animals will be sacrificed 2-4 weeks after treatment depending on the tumor burdens of each cell line model and the total number and total weight of tumors of each mouse will be examined.

### Example 16

### Confirmation of the Synergistic Effect of the Anti-HDGF Antibody in Combination with Other Therapeutic Agents

Based on the inventors' preliminary results set forth above, the antibody will be combined with chemotherapeutic agents that represent current standard of care in our current standard care for lung cancer patients. The following agents will be emphasized: cisplatin, gemcitabin, paclitaxel, doxorubicin, Tarceva®, and Avastin®. We will start with A549 subcutaneous model with a fixed dose of our anti-HDGF antibody and ½, of the mouse MTD of the chemotherapeutic agents and Tarceva®. Avastin® dose will be 100 μg per mouse/per injection, which is considered the optimal biologic dose. The experimental procedures will be similar as described previously except the use of combination therapy and oral administration of Tarceva®. Top two regimens (based on anti-tumor activity) will then be tested in additional tumor models.

A mathematical model and a statistical method will be used to analyze the effects of combination regimens in the animal models. The log of tumor volume will be fitted using a quadratic model in time. To avoid numerical difficulties, 1.0 will be added to each volume prior to analysis. Treatment and time will be entered as fixed effects, mouse as a random effect. Interaction terms will be entered between the treatment term and both the linear and quadratic terms in time. A number of covariance structures will be examined for each model. The final structure will be selected using the Akaike's Information Criterion (Bozdogan, 1987). Interaction between two treatments (A and B) will be defined in the following way. Given time t, the mean tumor volume in the control group is V₀(t); in treatment group A, V_{A}(t); in treatment group B, V_{B}(t); and in the combined treatment group A+B, V_{AB}(t). Let p_{A}(ft)=V_{A}(t)/V₀(t), where p_{A}(t) is the proportion in which the treatment A reduces tumor volume at time t and thus V_{A}(t)=p_{A}(t) V₀(t). Similarly, define p_{B}(t) and p_{AB}(t). Then, if there is no synergy between treatments A and B, p_{AB}(t)=p_{A}(t) p_{B}(t). For example, if the tumor volume in treatment group A falls to 80% and the tumor volume in treatment group B falls to 70% of that in the control group at time t, then, in the absence of synergy, the combined treatment should reduce the tumor volume to 56% of that in the control group. Under the foregoing definition, synergy may be present at one time point (t) and not at others. The inventors hypothesis is that the equation p_{AB}(t)=p_{A}(t) p_{B}(t) may be reformulated as a linear hypothesis suitable for testing by substituting for the definitions of P_{A}, P_{B}, and P_{AB}, Taking the logs of both sides of the equations yields ln V_{AB}=ln V_{A}+ln V_{B}−ln V₀. This may be tested using an appropriate contrast. All of the analyses will be performed using SAS procedure MIXED in SAS version 6.12 (SAS Institute Inc., Cary, N.C.).

### Example 17

### Confirmation that Anti-HDGF Antibodies Inhibit Metastasis

The preliminary studies discussed above demonstrate a role of HDGF in lung cancer invasion and metastasis, and that the antibody-based therapy can inhibit metastasis of NSCLC cells. To establish lung metastasis, female nu/nu mice will be injected intravenously via tail vein with 1×10⁶ A549 tumor cells suspended in 200 μl of sterile PBS. After 6 days, mice will be divided into groups (10 per group) and treated with M31, single anti-HDGF antibody, and the selected best combination in the dose optimized in earlier experiments. Animals will be sacrificed 3 weeks after initial treatment. Lungs from each of the mice from each group will be injected intratracheally with India ink and fixed in Feketes solution. The therapeutic effect of each group will be determined by counting the number of metastatic tumor nodules in each lung under a dissecting microscope without knowledge of the treatment group. The data will be analyzed and interpreted as statistically significant if the P-value is <0.05 by the Mann-Whitney rank-sum test. To determine the therapeutic effect of the treatment on animal survival, the mice 6 days after tumor cell injection will be divided into groups (10 per group) as described above. After a total of six doses of treatment, animals will be monitored daily for morbidity and mortality. Animals that were moribund will be euthanized by CO₂ inhalation. The therapeutic effects of the treatments will be determined by statistical analysis using the Kaplan-Meier survival estimation and Wilcoxon signed-rank sum tests.

### Example 18

### Confirmation that Anti-HDGF Therapy Promotes Tumor Growth Inhibition, Anti-Angiogenesis and Apoptosis

The initial studies discussed above demonstrated that anti-HDGF therapy promotes tumor growth inhibition, anti-angiogenesis and apoptosis (particularly in combination with secondary agents). It is proposed to carry out studies to confirm these initial findings.

For each tumor model described above, tumors will be collected from mice treated with different regimens. Tumor tissues from mice treated with effective regimens will be used to study the mechanisms of the anti-tumor activities of the regimens. This will include histopathologic examinations of the tumors and lungs (for orthotopic and metastasis models) to determine potential changes in tumor morphology and necrosis status by comparing results with controls and among treatment groups. Cell proliferation, apoptosis, and angiogenesis will be assessed in these tumors. For cell proliferation, BrdU labeling index will be studied by i.p. injection of BrdU 6 h before sacrifice of animals followed by BrdU immunohistochemistry detection. Briefly, 10 μm frozen sections will be fixed with ethanol and then washed in distilled water for 10 min and treated with 2 M HCl at room temperature for 1 h followed by neutralization for 5 min in 0.1 M sodium borate. Slides will be then washed in distilled water and transferred to a PBS bath. BrdU incorporated into DNA will be detected using a 1:200 dilution of monoclonal rabbit anti-BrdU followed by 1:100 dilution of anti-rabbit peroxidase conjugate antibody and 1:10 dilution of metal-enhanced 3,3′-diaminobenzidine substrate. Slides will be counterstained with hematoxylin, dehydrated, and mounted using Permount.

TUNEL assay will be used to determine apoptotic index. Because TUNEL assay recognizes DNA damages caused by other factors not limited to apoptotic process, expression levels of activated caspase-3, the primary executioner caspase, will be assessed because it cleaves other important proteins that are necessary to induce apoptosis. The activated caspase-3 protein will be detected immunohistochemically using a rabbit monoclonal antibody (5A1) (Cell Signaling); briefly, tissue sections are deparaffinized and rehydrated through graded alcohols. For antigen unmasking, slides will be boiled in 10 mM sodium citrate buffer pH 6.0 then maintain at a sub-boiling temperature for 10 minutes. After blocking, slides will be incubated with the primary antibody specific to the active caspase-3 in 1:200 dilutions for overnight at 4° C. The slides will than incubate for 30 minutes with biotinylated antirabbit immunoglobulin G (Vector Laboratories, Burlingame, Calif.). The slides will be then processed using standard avidin-biotin IHC according to the manufacturer's recommendations. Diaminobenzidine will be used as a chromogen, and commercial hematoxylin will be used for counterstaining. Number of TUNEL-positive cells and caspase-3 positive cells in 10 random 0.159 mm² fields at ×100 magnification will be used to quantify apoptosis.

To demonstrate the inhibition of angiogenesis, tumor tissue sections from treated groups and control groups will be assessed for microvessel density and expression of critical angiogenesis related markers. Double immunofluorescence staining for CD31 and VEGFR, pVEGFR, PDGFRβ, pPDGFRβ, pericytes (desmin-positive cells), and αSMA (smooth muscle cell marker) will be carried out. Briefly, frozen sections of the xenograft tumors will be mounted on slides and fixed. Immunofluorescence for CD31 will be done using Alexa 594-conjugated secondary antibody and samples will be blocked briefly in a blocking solution (5% normal horse serum and 1% normal goat serum in PBS) and incubated with antibodies against human VEGFR, pVEGFR, PDGFRβ, pPDGFRβ, or desmin at 4° C. overnight. After washes and blocking, samples will be incubated with Alexa 488-conjugated secondary antibody. Endothelial cells will be identified by red fluorescence and VEGFR, pVEGFR, PDGFRβ, pPDGFRβ, and desmin-positive cells (pericytes) will be identified by green fluorescence. The presence of growth factor receptors and phosphorylated receptors on endothelial cells will be detected by colocalization of red and green fluorescence, which appeared yellow. The coverage of pericytes on endothelial cells, will be determined by counting CD31-positive cells in direct contact with desmin-positive cells and CD31-positive cells without direct association with desmin-positive cells in five randomly selected microscopic fields (at ×200 magnification).

With respect to pathway markers and signaling, data from the initial studies set forth above demonstrate that HDGF may be involved in regulating a panel of genes including those critical in cell invasion and CEM formation. Analyzing expression of these molecules will provide not only knowledge about the regulation of HDGF-mediated down-stream signals but also potential modulations of CEM structures by the antibody-based regimens. Expression levels of SERPINE2, EGFR, HSPA5, PPPICC, AXL, GALNT1, NT5E, and NNMT using IHC and western blot analysis to determine effects of the regimens in expression of these proteins. Antibodies against these proteins are all commercially available.

From the studies carried out to date, it is anticipated that C1 and H3 monoclonal antibodies possess single agent activity in inhibiting tumor growth and prolong animal survival in both subcutaneous and orthotopic tumor models. All of the preclinical and clinical data support the role of HDGF in cancer invasion and metastasis. Therefore, it is contemplated that the antibody-based treatment, particularly combination regimen(s), will reduce or even eradicate lung cancer metastasis and prolong survival. As noted, based on the preclinical data, it is contemplated that the anti-tumor activity is achieved through a combinational growth inhibition, anti-angiogenesis, and disrupting tumor ECM formation. Therefore, it is contemplated that tumors treated with the antibody-based regimens may show a reduced BrdU incorporation, reduced or altered microvessels, and disrupted tumor stroma structures. Increased apoptosis will likely be observed in tumors, particularly those treated with combination regimens.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,659,774
U.S. Pat. No. 4,683,202
U.S. Pat. No. 4,816,571
U.S. Pat. No. 4,870,287
U.S. Pat. No. 4,959,463
U.S. Pat. No. 5,141,813
U.S. Pat. No. 5,214,136
U.S. Pat. No. 5,223,618
U.S. Pat. No. 5,264,566
U.S. Pat. No. 5,378,825
U.S. Pat. No. 5,428,148
U.S. Pat. No. 5,446,137
U.S. Pat. No. 5,466,786
U.S. Pat. No. 5,470,967
U.S. Pat. No. 5,539,082
U.S. Pat. No. 5,554,744
U.S. Pat. No. 5,574,146
U.S. Pat. No. 5,602,240
U.S. Pat. No. 5,602,244
U.S. Pat. No. 5,610,289
U.S. Pat. No. 5,623,070
U.S. Pat. No. 5,645,897
U.S. Pat. No. 5,652,099
U.S. Pat. No. 5,670,663
U.S. Pat. No. 5,672,697
U.S. Pat. No. 5,681,947
U.S. Pat. No. 5,686,072
U.S. Pat. No. 5,700,922
U.S. Pat. No. 5,705,629
U.S. Pat. No. 5,708,154
U.S. Pat. No. 5,714,331
U.S. Pat. No. 5,714,606
U.S. Pat. No. 5,719,262
U.S. Pat. No. 5,736,336
U.S. Pat. No. 5,760,395
U.S. Pat. No. 5,763,167
U.S. Pat. No. 5,766,855
U.S. Pat. No. 5,773,571
U.S. Pat. No. 5,777,092
U.S. Pat. No. 5,786,461
U.S. Pat. No. 5,786,461
U.S. Pat. No. 5,792,847
U.S. Pat. No. 5,858,988
U.S. Pat. No. 5,859,221
U.S. Pat. No. 5,872,232
U.S. Pat. No. 5,886,165
U.S. Pat. No. 5,891,625
U.S. Pat. No. 5,908,845
U.S. Pat. No. 5,891,625
U.S. Pat. No. 6,506,559
U.S. Pat. No. 6,573,099
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 4,938,948
U.S. Pat. No. 5,021,236
U.S. Pat. No. 5,196,066
U.S. Pat. No. 4,664,911
U.S. Pat. No. 4,682,195
U.S. Pat. No. 4,792,447
U.S. Pat. No. 5,045,451
U.S. Pat. No. 5,578,706
U.S. Pat. No. 5,767,072
U.S. application Ser. No. 117,363
U.S. Appln. 2002/0168707
U.S. Appln. 2003/0051263
U.S. Appln. 2003/0055020
U.S. Appln. 2003/0159161
U.S. Appln. 2004/0064842
U.S. Appln. 2004/0265839
Abbondanzo et al., Breast Cancer Res. Treat., 16:182(151), 1990.
Abouzied et al., J. Biol. Chem., 280(12):10945-10954, 2005.
Allred et al., Breast Cancer. Res. Treat., 16:182(149), 1990.
Atherton et al., Biol. of Reproduction, 32, 155-171, 1985.
Berberian et al., Science, 261:1588-1591, 1993.
Bernard et al., Cancer Res., 63(20):6716-6725, 2003.
Brown et al. Immunol. Ser., 53:69-82, 1990.
Buchholz et al., Cancer Res., 63:4945-51, 2003.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Cleary et al., Trends Microbiol., 4:131-136, 1994.
Culver et al., Science, 256(5063):1550-1552, 1992.
De Jager et al., Semin. Nucl. Med., 23(2):165-179, 1993.
Dholakia et al., J. Biol. Chem., 264, 20638-20642, 1989.
Doolittle and Ben-Zeev, Methods Mol. Biol., 109:215-237, 1999.
Egholm et al., Nature, 365(6446):566-568, 1993.
Elbashir et al., EMBO J., 20:6877-88, 2001.
Enomoto et al., Hepatology, 36:1519-1527, 2002.
Enomoto et al., Hepatology, 36:1519-27, 2002.
EP 266,032
Everett et al., Am. J. Physiol. Lung Cell Mol. Physiol., ₂₈₆:L1194-201, 2004.
Everett et al., J. Biol. Chem., 276:37564-8, 2001.
Everett et al., J. Clin. Invest., 105:567-75, 2000.
Everett, Dev. Dyn., 222:450-458, 2001.
Froehler et al., Nucleic Acids Res., 14(13):5399-5407, 1986.
Gefter, et al., Somatic Cell Genet., 3:231-236, 1977.
Goding, In: Monoclonal Antibodies: Principles and Practice, 2d ed., Academic Press, Orlando, Fla., pp 60-61, 71-74, 1986.
Goodman and Gilman's The Pharmacological Basis Of Therapeutics, Hardman et al. (Eds.), 10^{th} Ed., 32:853-860; 35:891-893, 2001.
Green, J. Immunol. Methods, 231(1-2):11-23, 1999.
Gulbis and Galand, Hum. Pathol, 24(12):1271-1285, 1993.
Harlow and Lane, In: Antibodies, a Laboratory Manual, Cold Spring Harbor Laboratory, 139-281, 1988.
Hu et al., Cancer, 98(7):1444-1456, 2003.
Iwasaki et al., Oncol Rep., 13:1075-80, 2005.
Jemal et al., Cancer J. Clin., 52:2347, 2002.
Kang et al., Science, 240:1034-1036, 1988.
Khatoon et al., Ann. of Neurology, 26, 210-219, 1989.
King et al., J. Biol. Chem., 269, 10210-10218, 1989.
Kishima et al., J. Biol. Chem., 277:10315-22, 2002.
Kohler and Milstein, Eur. J. Immunol, 6:511-519, 1976.
Kohler and Milstein, Nature, 256:495-497, 1975.
Kohler et al., Methods Enzymol., 178:3, 1989.
Kornberg and Baker, DNA Replication, 2nd Ed., Freeman, San Francisco, 1992.
Kreier et al., In: Infection, Resistance and Immunity, Harper & Row, New York, 1991.
Le Chevalier, Am. Soc. Clin. Oncol, 22:6, 2003.
Lenert et al., Science, 248:1639-1643, 1990.
Lepourcelet et al., Development, 132(2):415-427, 2005. Merck Index, 11^{th} Edition
Meric et al., Clin. Cancer Res., 8:361-7, 2002.
Mori et al., Am. J. Respir. Cell Mol. Biol, 30:459-69, 2004.
Nakamura et al., Clin. Chim. Acta, 183:273-84, 1989.
Nakamura et al., In: Handbook of Experimental Immunology (4^{th} Ed.), Weir et al., (eds). 1:27, Blackwell Scientific Publ., Oxford, 1987.
Nakamura et al., J. Biol. Chem., 269:25143-9, 1994.
Nakano et al., Clin. Exp. Metastasis, 20:665-74, 2003.
Non-small Cell Lung Cancer Collaborative Group, BMJ, 311:899-909, 1995.
Okuda et al., Cancer Sci., 94:1034-41, 2003.
Oliver and Al-Awqati, J. Clin. Invest., 102:1208-1219, 1998.
O'Shannessy et al., J. Immun. Meth., 99, 153-161, 1987.
Owens & Haley, J. Biol. Chem., 259:14843-14848, 1987.
Parkin et al., Cancer J. Clin., 49:33-41, 1999.
PCT Appln. PCT/EP/01219
PCT Appln. WO 92/20702
Physicians Desk Reference
Potter & Haley, Meth. in Enzymol., 91, 613-633, 1983.
Remington's Pharmaceutical Sciences, 18th Ed. Mack Printing Company, 1289-1329, 1990.
Remington's Pharmaceutical Sciences, 19th Ed. Mack Printing Company, 1995.
Remington's Pharmaceutical Sciences, 19th Ed. Mack Printing Company, 1995
Ren et al., J. Clin. Oncol., 22:3230-7, 2004.
Sakamoto et al., Clin. Cancer Res., 7:2513-8, 2001.
Sambrook et al., In: Molecular cloning, Cold Spring Harbor Laboratory Press, Cold Spring Harbor, N.Y., 2001.
Sasso et al., J. Immunol, 142:2778-2783, 1989.
Scheit, In: Synthesis and Biological Function, Wiley-Interscience, NY, 171-172, 1980.
Shorki et al., J. Immunol, 146:936-940, 1991.
Silvermann et al., J. Clin. Invest., 96:417-426, 1995.
Sue et al., J. Mol Biol, 343(5):1365-1377, 2004.
von Heijne, Nucleic Acids Res., 14:4683-90, 1986.
von Heijne, Nucleic Acids Res., 14:4683-90, 1986.
Willians et al., J. Thorac. Cardiovasc. Surg., 82:70-6, 1981.
Wimmel et al., Eur. J. Cancer, 37:2264-74, 2001.
Wu et al., Anticancer Res., 22:1071-8, 2002.
Yoshida and Yoshikawa, J. Gastroenterol., 38(15)15:31-34, 2003.
Zhang et al., Nat. Biotechnol, 21:818-21, 2003.
Jemal et al., CA Cancer J Clin 2006; 56:106-130.
Zhang et al., Cancer Res 2006; 66:18-23.
Nesbitt et al., Ann Thorac Surg 1995; 60:466-472.
Arriagada et al., N Engl J Med 2004; 350(4):351-360.
Crino et al., Seminars in Oncology 2002; 29:9-16.
Mao et al., Lung Cancer 2001; 34:S27-34.
Clark et al., Semin Oncol 1988; 15:20-5.
Barker et al., Curr Opin Investig Drugs 2003; 4:652-7.
Volpi et al., J Clin Oncol 2003; 21:2708-12.
Slamon et al., N Engl J Med 2001; 344:783-92.
Paez et al., Science 2004; 304(5676):1497-500.
Pastorino et al., J Clin Oncol 1997; 15(8):2858-65.
Qiu et al., Nat Struct Biol 2002; 9:217-24.
Stater et al., J Mol Biol 2003; 330:571-6.
Stec et al., FEBS Lett 2000; 473:1-5.
Izumoto et al., Biochem. Biophys. Res Commun 1997; 238: 26-32.
Ikegame et al., Biochem Biophys Res Commun 1999; 266: 81-87.
Abouzied et al., Biochem J 2004; 378: 169-176.
Dietz et al., Biochem J 2002; 366: 491-500.
Singh et al., Gene 2002; 242: 265-273.
Honegger et al., Cell 1987; 51:199-209.
Baldin et al., EMBO J. 1990; 9:1511-1517.
Matsuyama et al., Cancer Res 2001; 61:5714-7.
Bruchova et al., Leuk Lymphoma 2002; 43(6):1289-95.
Bernard et al., Cancer Res 2003; 63(20):6716-25.
Yamamoto et al., Clin Cancer Res 2006; 12(1):117-22.
Kishima et al., Hepatogastroenterology. 2002, 49:1639-44.
Perez-Soler et al., Clin Cancer Res 2001; 6(12):4932-8.
Lee et al., The American Statistician 1997; 51:353-358.
Lee et al., The American Statistician 2000; 54:63-70.
Therneau et al., Modeling survival data: extending the Cox model. 2000, New York: Springer-Verlag.
Bozdogan et al., Psychometrika 1987; 52: 345-370.

1. A method for reducing the growth of or inducing apoptosis in hyperproliferative cells in a subject comprising administering to said subject an amount of an HDGF-targeting agent that down regulates HDGF action that is effective to reduce the growth of or induce apoptosis in said cells.

2.-8. (canceled)

9. The method of claim 1, wherein the agent is a siNA or an antibody that recognizes HDGF.

10. The method of claim 9, wherein the HDGF-targeting agent is a siNA.

11.-12. (canceled)

13. The method of claim 9, wherein the HDGF-targeting agent is an antibody.

14.-36. (canceled)

37. The method of claim 1, wherein the hyperproliferative cell is a cancer cell.

38.-42. (canceled)

43. A method for assessing a cell suspected of being a cancer cell to determine the oncogenic potential of the cell, or of assessing the clinical outcome, prognosis, survival potential, aggressiveness or staging of a cancer patient, or diagnosis of a patient suspected of having a cancer, the method comprising assessing the HDGF expression of the cell or a sample obtained from the patient.

44.-58. (canceled)

59. A monoclonal antibody which binds and neutralizes HDGF.

60. The antibody of claim 59, wherein the antibody comprises SEQ ID NO: 32 or SEQ ID NO:36; and SEQ ID NO:34 or SEQ ID NO:38.

61. The antibody of claim 59, wherein the antibody is chimeric, humanized or human.

62. The antibody of claim 59, wherein the antibody selectively binds both a HDGF homolog and HDGF.

63. The antibody of claim 59, wherein the antibody is linked to an effector molecule or a reporter molecule.

64. The antibody of claim 63, wherein the antibody is linked to a reporter molecule.

65. The antibody of claim 64, wherein the reporter molecule is an enzyme, a radiolabel, a hapten, a fluorescent label, a phosphorescent molecule, a chemiluminescent molecule, a chromophore, a luminescent molecule, a photoaffinity molecule, a ligand, a colored particle or biotin.

66. The antibody of claim 63, wherein the antibody is linked to an effector molecule.

67. The antibody of claim 66, wherein the effector molecule is a toxin, an apoptotic molecule, an antitumor agent, a therapeutic enzyme or a cytokine.

68. The antibody of claim 59, wherein the antibody is comprised in a pharmaceutically acceptable carrier.

69. The antibody of claim 68, wherein the pharmaceutical composition is formulated for parenteral, intravenous or topical administration.

70. The antibody of claim 59, wherein the antibody is further defined as a C1 or H3 antibody.

71. An isolated nucleic acid encoding an amino acid sequence of an antibody in accordance with claim 59.

72. A composition comprising a siNA that reduces the expression of HDGF.

73. The composition of claim 72, wherein the siNA is an siRNA.

74. The composition of claim 72, wherein the siRNA comprises SEQ ID NO:47 or SEQ ID NO:48.

75.-82. (canceled)
